(19) United States
(12) Patent Application Publication
(10) Pub. No.: US 2023/0266279 A1
(43) Pub. Date: Aug. 24, 2023

(54) GAS ANALYSIS THROUGH SNIFFING SEQUENCES

(71) Applicant: President and Fellows of Harvard College, Cambridge, MA (US)

(72) Inventors: Sören BRANDT, Somerville, MA (US); Joanna AIZENBERG, Cambridge, MA (US); Venkatesh MURTHY, Cambridge, MA (US); Haritosh PATEL, Cambridge, MA (US)

(21) Appl. No.: 18/003,818

(22) PCT Filed: Jul. 1, 2021
(86) PCT No.: PCT/US2021/040126

§ 371 Date: Dec. 29, 2022

Related U.S. Application Data
(60) Provisional application No. 63/047,086, filed on Jul. 1, 2020.

(51) Int. Cl. G01N 30/16; G01N 30/74; G01N 33/497

(52) U.S. Cl. CPC G01N 30/16; G01N 30/74; G01N 33/497

(57) ABSTRACT

A method and device for analyzing a gas are described. A method for analyzing a gas includes introducing the gas into a chamber according to a sniffing recipe, the chamber including a sensor, wherein the sniffing recipe comprises a sequence of actions and the sniffing recipe is either pre-defined, optimized or determined through machine learning, and detecting, over time and by the sensor, a characteristic indicative of a compound or compounds present in the gas. The use of sniffing sequences can provide active, dynamic odor/gas identification with adaptive or self-optimizing capabilities.

### RELATED APPLICATION

This application claims the benefit of priority under 35 U.S.C. § 119(e) to co-pending U.S. Application Ser. No. 63/047,086, filed Jul. 1, 2020, the contents of which are incorporated in their entirety by reference.

### INCORPORATION BY REFERENCE

All patents, patent applications and publications cited herein are hereby incorporated by reference in their entirety in order to more fully describe the state of the art as known to those skilled therein as of the date of the invention described herein.

### FIELD OF THE DISCLOSURE

The present disclosure relates generally to the field of gas analysis. More particularly, the present disclosure relates to the analysis of compounds contained in a gas. In some embodiments, the analysis includes the use of sniffing sequences, which can provide active, dynamic odor/gas identification with adaptive or self-optimizing capabilities.

### SUMMARY

In one aspect, a method for analyzing a gas is described. The method includes:
providing a chamber comprising an inlet, an outlet and a sensor;
introducing the gas into the chamber;
controlling a concentration of the gas in the chamber according to a sniffing recipe, wherein the sniffing recipe comprises a sequence of actions and the sniffing recipe is either pre-defined, optimized or determined through machine learning,
wherein the sniffing recipe comprises:
(1) inhale, wherein inhale comprises introducing the gas into the chamber; and at least one of the following actions:
(2) exhale, wherein exhale comprises cleansing the sensor;
(3) wait, wherein wait comprises allowing a concentration of the gas to decrease slowly; and

detecting, over time and by the sensor, a characteristic indicative of a compound or compounds present in the gas.

In any one or more of the embodiments described herein, the sniffing recipe includes a pattern of actions.

In any one or more of the embodiments described herein, the sniffing recipe includes a specified length of time for each action in the sequence.

In any one or more of the embodiments described herein, sniffing recipe further includes one or more of the following actions: (4) hold, wherein hold comprises maintaining a relatively constant atmosphere in the chamber; (5) pressurize, wherein pressurize comprises increasing the pressure in the chamber; (6) convect, wherein convect comprises circulating the contents of the chamber; (7) de-pressurize or vacuum, wherein de-pressurize or vacuum comprises reducing the pressure in the chamber; (8) priming, wherein priming comprises introducing a known compound to the chamber before introducing the gas; (9) co-injection, wherein co-injection comprises introducing a known chaperone compound simultaneously with the gas; and (10) after-injection, wherein after-injection comprises introducing a compound that affects the desorption of the gas after introducing the gas to the chamber.

In any one or more of the embodiments described herein, the sniffing recipe includes a plurality of inhale actions alternating with a plurality of hold actions.

In any one or more of the embodiments described herein, the sniffing recipe includes a pattern of actions and a specified length of time for each action in the pattern, wherein the sequence of actions and specified length of time are pre-defined.

In any one or more of the embodiments described herein, the pre-defined pattern of actions is based on the gas being analyzed.

In any one or more of the embodiments described herein, the sniffing recipe comprises a first recipe followed by a second recipe, wherein the first recipe is pre-defined and the second recipe is determined based on machine learning from measurements resulting from the first recipe.

In any one or more of the embodiments described herein, the chamber is primed with a known compound prior to injecting the gas being analyzed.

In any one or more of the embodiments described herein, a known compound is co-injected simultaneously with the gas being analyzed.

In any one or more of the embodiments described herein, a known compound is injected after injecting the gas being analyzed.

In any one or more of the embodiments described herein, the sensor includes a photonic crystal.

In any one or more of the embodiments described herein, the sensor includes a field-effect transistor (FET).

In any one or more of the embodiments described herein, exhale includes flushing the chamber with another fluid to remove the gas being analyzed. In accordance with certain embodiments, flushing the chamber with another fluid includes injecting the fluid through the inlet.

In another aspect, a device is described, including:

a chamber configured to receive a gas to be analyzed, the chamber including an inlet and an outlet; a sensor disposed in the chamber, the sensor configured to detect a characteristic indicative of a compound or compounds present in the gas; and a pump configured to operate in accordance with a sniffing recipe, wherein the sniffing recipe comprises a sequence of actions and the sniffing recipe is either pre-defined, optimized, determined through machine learning, or a combination thereof, wherein the sniffing recipe comprises:

(1) inhale, wherein inhale includes activating the pump to introduce the gas into the chamber; and at least one of the following actions:

(2) exhale, wherein exhale includes flushing the chamber to remove the gas

(3) wait, wherein wait comprises allowing a concentration of the gas to decrease slowly.

In any one or more of the embodiments described herein, the sniffing recipe further includes one or more of the following actions: (4) hold, wherein hold comprises holding the gas in the chamber; (5) pressurize, wherein pressurize comprises increasing the pressure in the chamber to sample a larger section of the adsorption isotherm; (6) convect, wherein convect comprises circulating the contents of the chamber; (7) de-pressurize or vacuum, wherein de-pressurize or vacuum comprises reducing the pressure in the chamber; (8) priming, wherein priming comprises introducing a known compound to the chamber before introducing the gas; (9) co-injection, wherein co-injection comprises introducing a known chaperone compound simultaneously with the gas; and (10) after-injection, wherein after-injection comprises introducing a compound that affects the desorption of the gas after introducing the gas to the chamber.

In any one or more of the embodiments described herein, the sensor is selected from the group consisting of a photonic crystal, a field effect transistor, a nanogenerator, and photomechatronic nanostructures.

In any one or more of the embodiments described herein, the sensor is a photonic crystal.

In any one or more of the embodiments described herein, the sensor provides a spectral response.

In any one or more of the embodiments described herein, the spectral response comprises a bandgap shift.

In any one or more of the embodiments described herein, the device further includes a spectrophotometer configured to detect the evolution of the spectral response in time.

In any one or more of the embodiments described herein, the device further includes at least one processor configured to run one or more machine learning algorithms on data provided by the sensor, the machine learning algorithm capable of determining a pattern of actions based on features of the data from the sensor, wherein at least one of the one or more machine learning algorithms comprises at least one of pattern recognition, classification, regression, and segmented regression.

In any one or more of the embodiments described herein, the one or more machine learning algorithms are selected from the group consisting of LASSO, kernel ridge regression, decision trees, bagging classifiers, multiclass logistic regression, principle component analysis, linear discriminant analysis, supervised machine learning, semi-supervised machine learning, non-supervised machine learning, support vector machines, transfer learning neural networks, segmented regression, and a combination thereof.

In any one or more of the embodiments described herein, the pump or a second pump is configured to inject a known compound(s) into the chamber in accordance with one or more of the following:

1) prior to introducing the gas being analyzed;

2) simultaneously with the gas being analyzed;

3) after introducing the gas being analyzed.

In any one or more of the embodiments described herein, the pump or a second pump is configured to introduce a known compound(s) into the chamber in accordance with one or more of the following:

1) prior to introducing the gas being analyzed;

2) simultaneously with the gas being analyzed;

3) after introducing the gas being analyzed.

In any one or more of the embodiments described herein, the device may also include a filter disposed in the chamber between the inlet and the sensor. In accordance with certain aspects, the filter includes a size exclusive mesh.

In any one or more of the embodiments described herein, the device is selected from the group consisting of an indoor sensor, a medical diagnostic device, a food quality sensor, an air quality sensor and combinations thereof.

In any one or more of the embodiments described herein, the gas being analyzed may be from a biological sample, such as a person, animal, food item, etc.

In another aspect, a device is described, including a chamber configured to receive a gas to be analyzed, wherein the chamber includes an inlet and, in some cases, an outlet. A sensor is disposed in the chamber, wherein the sensor is configured to detect a characteristic indicative of a compound or compounds present in the gas. The device is configured to operate in accordance with a sniffing recipe, wherein the sniffing recipe includes a sequence of actions and the sniffing recipe is either pre-defined, optimized, determined through machine learning, or a combination thereof, wherein the sniffing recipe includes:

(1) inhale, wherein inhale includes introducing the gas into the chamber; and at least one of the following actions:

(2) exhale, wherein exhale includes flushing the chamber to remove the gas;

(3) wait, wherein wait comprises allowing a concentration of the gas to decrease slowly.

In any one or more of the embodiments described herein, the device is a handheld device. In accordance with some embodiments, the device is a breathalyzer, smart phone or smart watch.

In any one or more of the embodiments described herein, the gas being analyzed may be a user's breath. In accordance with certain aspects, the device may provide instructions to the user to breathe in accordance with the sniffing recipe.

Any aspect or embodiment disclosed herein may be combined with another aspect or embodiment disclosed herein. The combination of one or more embodiments described herein with other one or more embodiments described herein is expressly contemplated.

Unless otherwise defined, used, or characterized herein, terms that are used herein (including technical and scientific terms) are to be interpreted as having a meaning that is consistent with their accepted meaning in the context of the relevant art and are not to be interpreted in an idealized or overly formal sense unless expressly so defined herein.

Although the terms, first, second, third, etc., may be used herein to describe various elements, these elements are not to be limited by these terms. These terms are simply used to distinguish one element from another. Thus, a first element, discussed below, may be termed a second element without departing from the teachings of the exemplary embodiments. Spatially relative terms, such as “above,” “below,” “left,” “right,” “in front,” “behind,” and the like, may be used herein for ease of description to describe the relationship of one element to another element, as illustrated in the figures. It will be understood that the spatially relative terms, as well as the illustrated configurations, are intended to encompass different orientations of the apparatus in use or operation in addition to the orientations described herein and depicted in the figures. For example, if the apparatus in the figures is turned over, elements described as “below” or “beneath” other elements or features would then be oriented “above” the other elements or features. Thus, the exemplary term, “above,” may encompass both an orientation of above and below. The apparatus may be otherwise oriented (e.g., rotated 90 degrees or at other orientations) and the spatially relative descriptors used herein interpreted accordingly. Further still, in this disclosure, when an element is referred to as being “linked to,” “on,” “connected to,” “coupled to,” “in contact with,” etc., another element, it may be directly linked to, on, connected to, coupled to, or in contact with the other element or intervening elements may be present unless otherwise specified.

The terminology used herein is for the purpose of describing particular embodiments and is not intended to be limiting of exemplary embodiments. As used herein, singular forms, such as “a” and “an,” are intended to include the plural forms as well, unless the context indicates otherwise.

### BRIEF DESCRIPTION OF THE DRAWINGS

The invention is described with reference to the following figures, which are presented for the purpose of illustration only and are not intended to be limiting. In the Drawings:

FIG. 1A shows a schematic illustration of a device for analyzing a gas, according to one or more embodiments. FIG. 1A also shows examples of injection sequences and various actions.

FIG. 1B shows a schematic illustration of an analysis process to progress from one sequence to another, according to one or more embodiments.

FIG. 1C shows a schematic illustration of a various types of sequences, according to one or more embodiments.

FIG. 1D lists some non-limiting examples for potential applications of an adaptive sensor as disclosed herein.

FIG. 2A shows a schematic of adsorption/desorption in time, according to one or more embodiments.

FIG. 2B shows adsorption/desorption kinetics under different conditions, according to one or more embodiments.

FIG. 2C shows competitive adsorption kinetics for two compounds, according to one or more embodiments.

FIG. 2D shows schematic of dynamic sniffing of biological samples in which the signature gas mixture is time evolving.

FIG. 3A shows plots for concentration v. time for various actions and shows the corresponding adsorption isotherm, according to one or more embodiments.

FIG. 3B shows plots for concentration v. time for various actions and shows the corresponding adsorption isotherm, according to one or more embodiments.

FIG. 4A shows a sniffing pattern and the resulting phase curves for hexane and ethanol, according to one or more embodiments.

FIG. 4B shows another sniffing pattern and the resulting phase curves for hexane and ethanol, according to one or more embodiments.

FIG. 4C shows yet another sniffing pattern and the resulting phase curves for hexane and ethanol, according to one or more embodiments.

FIG. 4D shows another sniffing pattern and the resulting phase curve for ethanol, according to one or more embodiments.

FIG. 5 shows phase curves for pentane and ethanol when subjected to various sniffing patterns, according to one or more embodiments.

FIG. 6A shows phase curves for various compounds when subjected to a short sniffing pattern, according to one or more embodiments.

FIG. 6B shows phase curves for various compounds when subjected to a deep sniffing pattern, according to one or more embodiments.

FIG. 6C shows phase curves for various compounds when subjected to a short sniffing pattern held, according to one or more embodiments.

FIG. 6D shows phase curves for various compounds when subjected to a short sniff, long exhale pattern, according to one or more embodiments.

FIG. 7A shows phase curves for various compounds when subjected to different sniff patterns, according to one or more embodiments.

FIG. 7B shows phase curves for various compounds when subjected to different sniff patterns, according to one or more embodiments.

FIG. 8A shows a confusion matrix for the results in FIG. 6A.

FIG. 8B shows a confusion matrix for the results in FIG. 6B.

FIG. 9A shows a phase signal for low-concentration toluene in water mixtures using a short sniff, long exhale sniffing sequence, according to one or more embodiments.

FIG. 9B shows a phase signal for low-concentration toluene in water mixtures using a short sniff, exhale sniffing sequence, according to one or more embodiments.

FIG. 10 shows phase signals for low-concentration toluene in water mixtures using two sniffing sequences with a different baseline gas containing water vapor to isolate the signal for toluene, according to one or more embodiments.

FIG. 11A shows a sensor device wherein solenoid valves and pressurized gas are used to inject gases according to sniffing recipes or patterns of actions, according to one or more embodiments.

FIG. 11B shows a sensor device wherein solenoid valves, pressurized gas and liquid filled tubes are used to inject gases according to sniffing recipes or patterns of actions, according to one or more embodiments.

FIG. 11C shows another arrangement for a sensor device wherein solenoid valves, pressurized gas and liquid filled tubes are used to inject gases according to sniffing recipes or patterns of actions, according to one or more embodiments.

FIG. 11D shows yet another arrangement for a sensor device wherein solenoid valves, pressurized gas and liquid filled tubes are used to inject gases according to sniffing recipes or patterns of actions, according to one or more embodiments.

FIG. 12A shows a signal sensitivity comparison for short sniff and deep sniff showing the diverse reaction to fast and slow sniffing sequences, according to one or more embodiments.

FIG. 12B shows an analysis of short sniff and deep sniff sequences on apolar and polar chemicals using support vector machine classification (machine learning), according to one or more embodiments.

FIG. 12C shows a detailed comparison of increased sensitivity of different sniffing sequences for apolar chemicals using short and deep sniff, according to one or more embodiments.

FIG. 13 shows a binary mixture concentration analysis using support vector regression, according to one or more embodiments.

FIGS. 14A and 14B show concentration analysis of binary mixtures of ethanol and water using a short sniff sequence and deep sniff sequence, respectively. These results show that the use of sniffing techniques can significantly improve the accuracy of the sensor, according to one or more embodiments.

FIG. 14C shows an analysis of the mixing ratio of binary mixtures of pentane-hexane, according to one or more embodiments.

FIG. 14D shows an analysis of the mixing ratio of binary mixtures of pentane-octane, according to one or more embodiments.

FIG. 15 shows a physical realization in which the sensing element can be of various forms (including acoustic, optical, or chemiresistive), and the sensing output can be the detection of various categories including but not limited to diseases/illnesses detection (such as cancers, malaria, or viruses), air quality inspection, or monitoring of illicit drugs.

FIG. 16 shows an indoor sensor which may include various sensing elements in which it may be installed in locations of residential homes, industrial sites, or institutional buildings for detection of air quality, airborne pathogens, and health monitoring.

FIG. 17 shows the application of sniffing technology to biological samples (foods). These samples can be sensed by either traditional sensors, smart mobile devices, packaging, or smart refrigerators to detect spoilage, food lifetime, and bacterial profile.

### DETAILED DESCRIPTION

There are impressive commercial sensors that mimic and outperform sight, hearing, and touch, but none that rival the sense of smell. In fact, the state-of-the-art in smell sensing is the use of dogs, which have been commonly trained for years to find drugs, explosives, and even diseases like cancer and potentially Parkinson's and Alzheimer's. Therefore, to improve man-made sensors and better mimic a nose, there is also a need to actively and quickly sample for vapors to search for identifying odors.

The present disclosure, in one or more embodiments, provides devices and methods for the detection and characterization of a gas and gas mixtures, and components thereof, using a pattern of actions that in some cases simulate sniffing, and utilizing stimuli-responsive sensors, such as photonic crystals or field-effect-transistors (FETs). Vapors of a sample or liquid can also be analyzed in accordance with the disclosed method. The methods and devices disclosed herein can also be used to analyze odor, fumes, liquid sprays, and aerosols, from biological and non-biological sources. As used herein, the term “gas” also encompasses these other types of samples that can be analyzed. The present disclosure in certain embodiments, simulates or incorporates aspects of the active sampling seen in the sniffing behaviors of dogs and other mammals to predict the properties of both liquids and gases in a diversity of applications, such as environmental (e.g., pesticide control) and medical monitoring (e.g., blood or urine). The use of sniffing sequences as disclosed herein, as opposed to existing static methods, provides active, dynamic odor/gas identification with adaptive or self-optimizing capabilities.

The use of sniffing sequences as disclosed herein, can also be used to evaluate dynamic samples that may change over time or during different test conditions. For example, the disclosed methods can be used to monitor and analyze biological samples. In some embodiments, the methods can be used to measure kinetics of biological systems such as bacterial systems wherein the changing systems can be sensed, classified, and regressed. The methods disclosed herein may be particularly useful for applications such as sensing biological growth signatures (providing a quantitative and/or qualitative analysis of bacteria), applications of food quality sensing, determining the kinetics and dynamics of bacterial growth and making inferences of environmental variables based on biological changes. In accordance with some embodiments, the sniffing sequences as disclosed herein can be used to analyze biological samples in which the gas analytes being sensed themselves are evolving from the sample due to various biological processes. This method can be used to understand the biological signature of such samples using machine learning and can undergo classification of bacterial make-up of samples, kinetics of bacterial growth—in cases where the substrate is a sample of food, the classification can be used to determine the quality of the substrate (such as healthiness/spoilage of food).

In terms of assessing the properties of the gas or gas mixtures being analyzed, approaches set forth in one or more embodiments herein provide several advantages when compared to other methods. For example, in some embodiments, the design of the architecture of, for example, photonic sensors or field effect transistor (FET) sensors allows for their unique gas sorption behavior to be exploited, and, thus, enable their application for a discriminative analysis of the compounds in the gas.

### Analyzing a Gas or Gas Mixtures

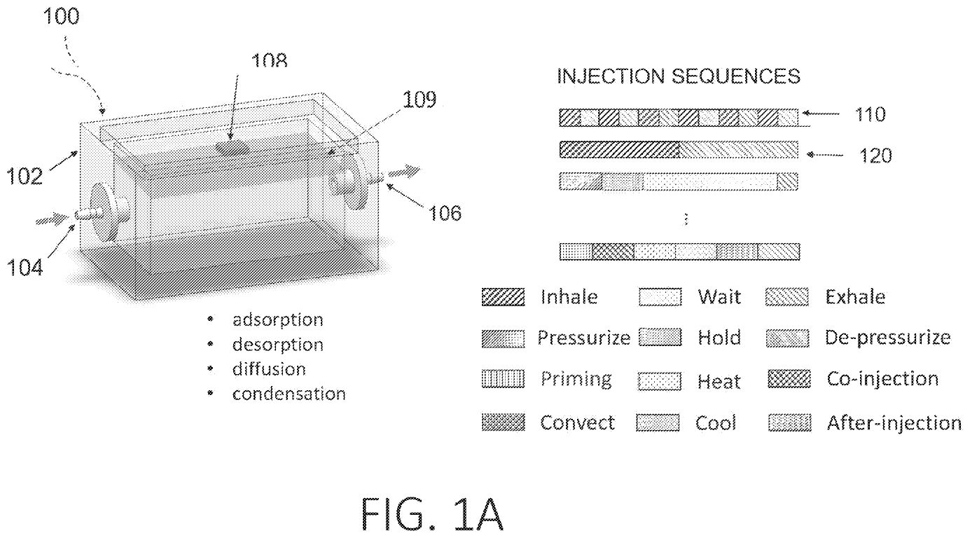

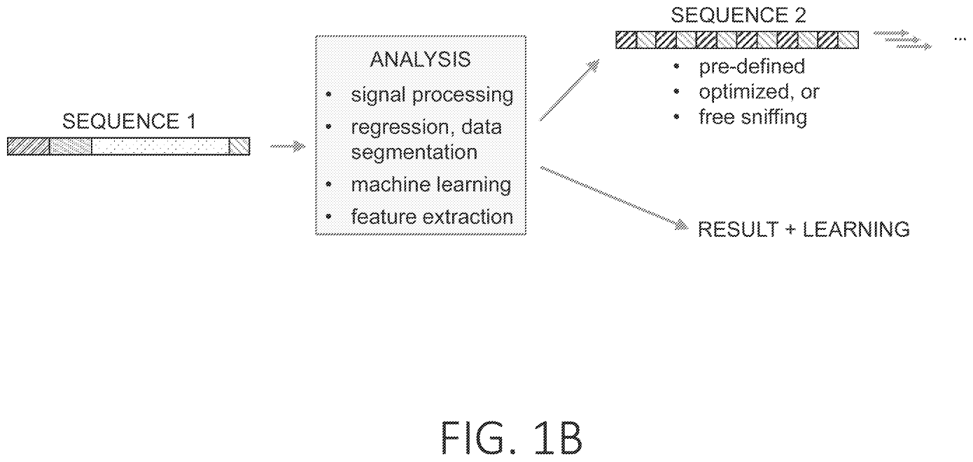

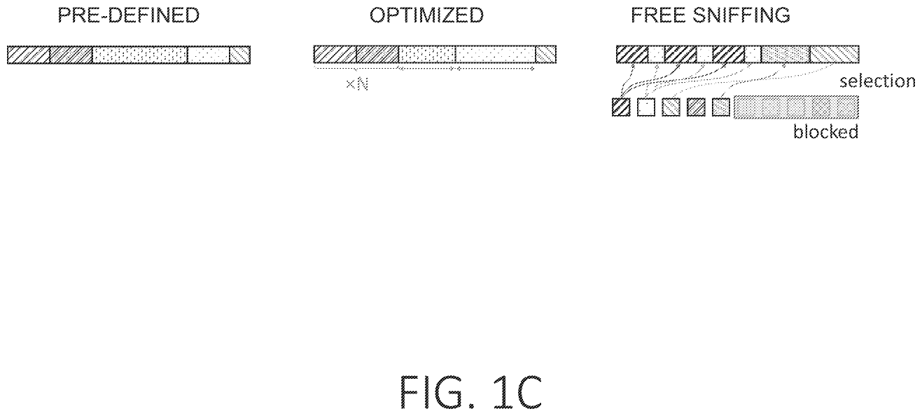

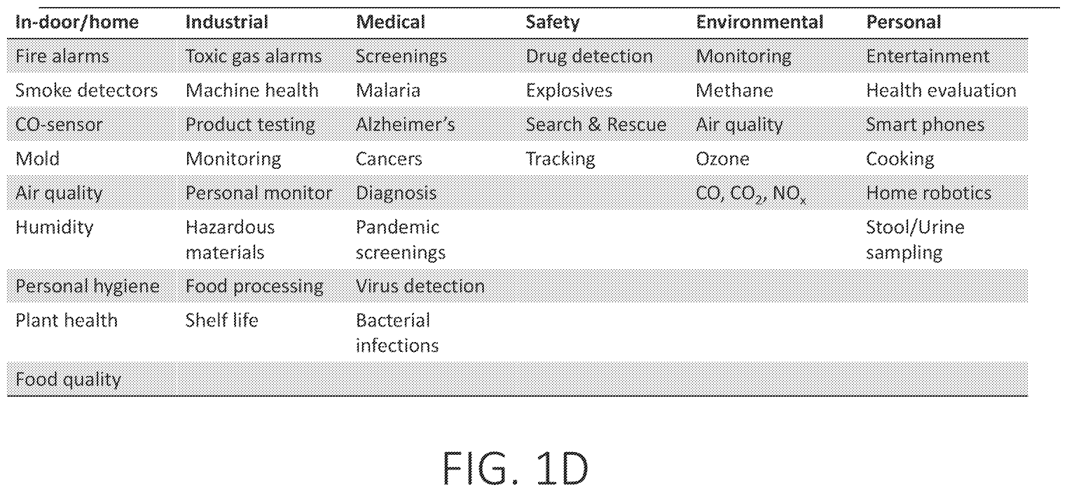

As shown in FIG. 1A, a device 100 includes, in some embodiments, a chamber 102 having an inlet 104 to transport a gas or gas mixture from outside the chamber to inside the chamber. In certain embodiments, inlet 104 is used to pressurize the chamber. Chamber 102 also includes an outlet 106 to transport a gas or gas mixture from inside of the chamber to the outside of the chamber. In some embodiments, the chamber may have a single opening capable of functioning as either an inlet or outlet depending on the situation. In some embodiments, the chamber may have multiple inlets and/or outlets. Flow through the inlet and outlet may be controlled by valves in fluid connection with each of the inlet and outlet. In some embodiments, the device 100 includes a sensor 108 disposed inside the chamber 102. The sensor is capable of detecting a plurality of features of the gas or gas mixture and how they change over time, providing a unique signature for the gas or gas mixture. In some embodiments, chamber 102 may further include a filter 109 to facilitate selective sniffing. In accordance with one aspect, filter 109 can remove the compound(s) responsible for the dominant odor in order to facilitate detection of other constituents of the composition.

In accordance with some embodiments, a gas or mixture of gasses for analysis is injected into, introduced into, removed from, or modified in a chamber containing a sensor using a sequence of sniffing steps, also referred to herein as actions. Using these sniffing steps, the gasses in the chamber can be changed through inhale, exhale, wait, hold, pressurize, convect or de-pressurize steps that control one or more of the various actions and properties, such as gas flow into and out of the chamber, concentration of gas in the chamber, the conditions in the chamber, opening and closing of the inlet and outlet valves. Inhale comprises introducing a sample gas to the chamber. In accordance with one aspect, the sample gas is introduced into the chamber through the inlet. In accordance with certain aspects, inhale comprises injecting the sample gas through the inlet and into the chamber. In accordance with some embodiments, the inlet and outlet to the chamber are both open. As a result, the concentration of the analyte in the chamber increases without intentionally also increasing the pressure inside the chamber. Exhale comprises cleansing the sensor. In accordance with one aspect, the sensor is cleaned by flushing the chamber to remove the sample gas. In accordance with one aspect, exhale comprises injecting a baseline gas or mixture of gasses (such as dry air) to flush the chamber to remove the sample gas. In accordance with some aspects, the inlet and outlet are both open such that the sample gas concentration quickly decreases. This procedure can also be used to flush the chamber before each analysis to ensure that the sensor, such as the photonic crystal—e.g., the Bragg stack—is clean. Wait comprises allowing the sample gas concentration to decrease slowly. In accordance with one aspect, wait comprises closing the inlet without injecting any gas into the chamber but leaving the outlet open such that the sample gas concentration decreases slowly over time. Hold comprises maintaining a relatively constant atmosphere in the chamber. In accordance with one aspect, hold comprises closing the inlet and outlet to keep a relatively constant atmosphere in the chamber. Pressurize comprises increasing the pressure in the chamber to sample a larger section of the adsorption isotherm. In accordance with one aspect, pressurize comprises opening the inlet to a sample gas while keeping the outlet closed to increase the pressure in the chamber to sample a larger section of the adsorption isotherm. This could allow one to probe specific odors and interactions at elevated pressures and concentrations. FIG. 1A provides one example of a sniffing sequence including a pressurize step 130. Convect comprises circulating the contents of the chamber. In accordance with one aspect, convect comprises circulating the contents of the chamber with both the inlet and outlet closed. In accordance with one embodiment, the convect action includes the use of fan-like mechanical device to stir the air within the chamber with both the inlet and outlet closed. This can be useful to facilitate movement of low diffusing gas analytes to the sensing area faster and within a reasonable timescale. De-pressurize or vacuum action comprises reducing the pressure in the chamber. In accordance with one aspect, vacuum can be produced by having the inlet closed or partially closed and the outlet open with a vacuum attached to the outlet or to the whole chamber. In accordance with some aspects, the vacuum action can provide for a lower concentration of gas in the chamber. Reducing the pressure in the chamber may operate to change physical dynamics of certain samples such as evaporation rate, partial pressure, and physical properties (such as flash point, etc.) of liquid samples. This can be useful for analyzing samples in which evaporation is a rate-limiting step in which the sensor is unable to detect the sample in an appropriate timescale since the sample is unable to volatilize the gas from the liquid state.

Furthermore, sniffing recipes could include combinations of sniffing features or actions to provide additional functionality to the system. For example, inhales for two compounds could be combined (either sequentially or at the same time), varying the mixing ratio to probe the competitive or combined adsorption of one known and one unknown species. Additional sniffing steps or actions can also be used to modify the behavior inside the chamber from the outside by heating, cooling, magnetic fields, ionization, or by introducing a known or unknown second gas or vapor before, during, or after injection of the sample to modify the pattern of gasses reaching the sensor and the sensor response. In some embodiments, Priming the sensor with a known compound before the introduction or injection of the tested mixture is described. Such step may promote the adsorption of the tested species or prevent the adsorption of the unwanted species in the gas mixture. In some embodiments, Co-injection of the tested gas or gas mixture with a known chaperone compound can take place simultaneously. The chaperone molecule can bind to a tested gas species, thus facilitating its adsorption and affecting its adsorption/desorption kinetics, or bind to certain components of the gas mixture, and creating species that are prevented from adsorption. In some embodiments, an After-injection step is used to inject a compound that affects the desorption step of an unknown gas or gas mixture. The steps of co-injection and after-injection also encompass introducing the compound or gas simultaneously or subsequently, respectively.

Sniffing recipes may include various sniffing steps in a sequence, which may have different durations, and take place at different pressures, or temperatures. Sniffing offers a potentially endless combination to dynamically modify the sensing without the need to replace parts of the sensor. Sniffing steps can be combined into any sequence to control the flow of gasses or vapors into and through the chamber and produce unique signatures to characterize the character (e.g., dangerous or not, healthy or sick, etc.), composition, or physical properties of the samples. In certain embodiments, as shown in FIG. 1A, a short sniff sequence 110, which is composed of a number of short inhale-exhale steps, is used. In certain embodiments, as shown in FIG. 1A, a deep sniff sequence 120, which is composed of a number of long inhale-exhale steps, is used. The use of these sniffing steps in a sequence as described above provides active, dynamic odor/gas identification with adaptive and/or self-optimizing capabilities that are not obtained with static methods.

### Chamber

The dimensions and materials of the chamber can be modified to limit and/or promote the flow of gasses and or the adsorption, desorption, diffusion, and condensation of gasses onto the surface or specific segments of the surface, including introducing other materials such as drying agents, porous materials, liquids, or others.

In some embodiments, the chamber can include glass, Teflon, or other solvent-resistant materials.

In some embodiments, one or more inside surfaces of the chamber 102 can have the same or different homogeneous or heterogeneous chemical patterns on the nanoscale and microscale.

In some embodiments, one or more inside surfaces of the chamber 102 can have the same or different homogeneous or heterogeneous topography patterns on the nanoscale and microscale.

In some embodiments, the surface of the inside of the chamber can be a hierarchical surface containing surface features on multiple length scales. In accordance with one aspect, the surface at the bottom of the inside of the chamber can be a hierarchical surface containing surface features on multiple length scales. For example, in some embodiments, the surface can have a first topological feature having dimensions on the microscale and a second topological feature on the nanoscale. In these embodiments, the first topological feature supports the second smaller topological feature. In some embodiments, the second topological features are referred to as “primary structures” as they are meant to denote the smallest feature sizes of the hierarchical structure. In these embodiments, the primary structures can include structures, such as nanofibers, or nanodots. In these embodiments, such nanoscale “primary structures” can have at least one kind of feature sizes that are a few to tens or hundreds of nanometers in size, such as less than 5 nm to 200 nm. For example, in these embodiments, nanofibers can have diameters of approximate 5, 10, 25, 50, or 100 nm. In some embodiments, in such cases, when “primary structures” having feature sizes of about 100 nm diameter are utilized, “secondary structures” having feature sizes that are larger than 100 nm, such as 150 nm, 300 nm, 500 nm, or 1000 nm, and larger can be utilized. Additional higher order structures, such as “tertiary structures,” each of which can have larger feature sizes than the lower order structures, are used in some embodiments.

In some embodiments, the chamber base has flat, round rectangular, square, triangular, or a geometrically complex shape with an area ranging from 1 mm² to 10000 mm². In some embodiments, the chamber has a height between about 0.1 cm to about 100 cm, more particularly between about 1 cm to about 30 cm.

In these embodiments, the homogeneous or heterogeneous chemical or topography patterns of one or more inside surfaces of the chamber 102 can tune the selectivity and sensitivity of the device for analyzing a gas or gas mixtures. For example, inclusion of pores or channels of various sizes on one or more surfaces of the chamber 102 can alter properties (e.g., kinetics) of wetting, evaporation, diffusion, or convection based on some analytes being able to enter the pores/channels (e.g., due to molecular size) and some not. Similarly, in some embodiments, inclusion of chemical coatings on one or more surfaces of the chamber 102 can alter properties (e.g., kinetics) of wetting, evaporation, diffusion, or convection based on intermolecular interactions between some analytes and said chemical coatings, but not other analytes. Thus, in these embodiments, for a given gas or gas mixture, the time it takes for certain analytes to reach the sensor can be altered via these chemical and topological modifications, thereby selecting detection of one or more analytes over one or more other analytes.

For example, in some embodiments, the one or more inside surfaces can be functionalized with silyl groups. Non-limiting examples of such silyl groups include perfluorooctyltrichlorosilane, triethoxsilylbutyraldehyde, bis(2-hydroxyethyl)-3-aminopropyltriethoxysilane, 3-chloropropyltriethoxysilane, 3-(trihydroxysilyl)-1-propanesulfonic acid, n-(triethoxysilylpropyl)-alpha-poly-ethylene oxide urethane, n-(trimethoxysilylpropyl)ethylene diamine triacetlc acid, n-octyltriethoxysilane, n-octadecyltriethoxysilane, (3-trimethoxysilylpropyl)diethylenetriamine, methyltriethoxysilane, hexyltrimethoxysilane, 3-aminopropyltriethoxysilane, hexadecyltriethoxysilane 3-mercaptopropyltrimethoxysilane, and dodecyltriethoxysilane, or chiral functionalities including N-(3-triethoxysilylpropyl)gluconamide or (R)—N-triethoxysilylpropyl-O-quinineurethane). In some embodiments, the one or more inside surfaces can be a roughened by including a porous material. In these embodiments, the roughened surface includes both the surface of a three-dimensionally porous material as well as solid surface having certain topographies, whether they have regular, quasi-regular, or random patterns. In some embodiments, the surface can be roughened by incorporation of micro textures. In other embodiments, the substrate can be roughened by incorporation of nano textures.

In some embodiments, microparticles or nanoparticles are applied to the surface to form a roughened, porous surface. In these embodiments, microparticles or nanoparticles can be applied to the surface using photolithography, projection lithography, electron-beam writing or lithography, depositing nanowire arrays, growing nanostructures on the surface of a substrate, soft lithography, replica molding, solution deposition, solution polymerization, electropolymerization, electrospinning, electroplating, vapor deposition, layered deposition, rotary jet spinning of polymer nanofibers, contact printing, etching, transfer patterning, microimprinting, self-assembly, boehmite formation, spray coating, and combinations thereof.

In some embodiments, the surface can include a fluoropolymer. Non-limiting examples of fluoropolymers can include polytetrafluoroethylene, polyvinylfluoride, polyvinylidene fluoride, and fluorinated ethylene propylene.

In some embodiments, the surface can include a plurality of holes, a three-dimensionally interconnected network of holes, or random array of fibrous materials.

In some embodiments, the roughened surface can be formed over a two-dimensionally flat surface by providing certain raised structures or protrusions. In other embodiments, the roughened surface can be formed by forming pores over a two-dimensionally flat surface to yield a porous material. In these embodiments, pores can have any geometry and can include pathways, columns, or random patterns. In yet other embodiments, a three-dimensionally interconnected network of regular or random pores is used, which can include open-cell bricks, post arrays, parallel grooves, open porosity PTFE (ePTFE), plasma-etched PTFE, and sand-blasted polypropylene (PP).

In certain embodiments, the roughened surface may have a periodic array of surface protrusions (e.g., posts or peaks) or any random patterns or roughness. In some embodiments, the size of the features producing the roughened surface can range from 10 nm to 100 μm, with geometries ranging from regular posts or open-grid structures to randomly oriented spiky structures. In some embodiments, the features can range from be any combination of low and high values of 10 nm, 25 nm, 50 nm, 100 nm, 250 nm, 500 nm, 1 m, 2.5 μm, 5 μm, 10 μm, 25 μm, 50 μm, or 100 am. In some embodiments, the widths of the raised structures can be constant along their heights. In some embodiments, the widths of the raised structures can increase as they approach the basal surface from the distal ends. In some embodiments, the raised structures can be raised posts of a variety of cross-sections, including, but not limited to, circles, ellipses, or polygons (e.g., triangles, squares, pentagons, hexagons, octagons, and the like), forming cylindrical, pyramidal, conical, or prismatic columns. Although the exemplary substrates described in these embodiments illustrate raised posts having uniform shape and size, the shape, orientation or size of raised posts on a given substrate can vary.

In some embodiments, a range of surface structures with different feature sizes and porosities can be used. In these embodiments, feature sizes can be in the range of hundreds of nanometers to microns (e.g., 50 to 1000 nm), and have aspect ratios from 1:1 to 10:1, from 1:1 to 2:1, from 1:1 to 3:1, from 1:1 to 4:1, from 1:1 to 5:1, from 1:1 to 6:1, from 1:1 to 7:1, from 1:1 to 8:1, and from 1:1 to 9:1. In some embodiments, porous nano-fibrous structures can be generated in situ on the inner surfaces of metallic microfluidic devices using electrochemical deposition techniques.

### Sensor

In some embodiments, the adsorption-desorption kinetics of vapors or gasses onto the sensor is the primary means to produce a sensor response. The evolution of the resulting time-dependent signal can be analyzed using machine learning. Examples of particularly useful sensors include, but are not limited to, a photonic crystal, a field effect transistor, a nanogenerator, photomechatronic nanostructures, light dependent resistor (LDR), photodiode, photo-transistor, solar cell, and chemiresistor sensors.

In some embodiments, the sensor can be on the side or at the end of a microfluidic channel on or in one or more surfaces of the chamber.

In some embodiments, microporous sensing materials for photonic, field effect transistor (FET), or nanogenerator-based sensors can include metal-organic framework (MOF) materials. In these embodiments, metal-organic framework (MOF) materials can be crystalline compounds consisting of rigid organic molecules held together and organized by metal ions or clusters (e.g., ZIF-8, CAU, and HKUST). In some embodiments, the metal organic framework (MOF) materials can include surface-mounted metal-organic frameworks (SURMOFs), iso-reticular metal-organic frameworks (IRMOFs), covalent organic framework (COF), zeolitic inorganic framework (ZIF), or a combination thereof. In some embodiments, the metal-organic framework (MOF) material is a porous material. In some embodiments, the metal-organic framework (MOF) materials can be functionalized to bind and interact with various volatile analytes including, but not limited to, ammonia, carbon dioxide, carbon monoxide, hydrogen, amines, methane, oxygen, argon, nitrogen, argon, organic dyes, polycyclic organic molecules, and combinations thereof. In some embodiments, the metal organic framework (MOF) materials can include a chemically-sensitive resistor, where the metal organic framework (MOF) material is disposed in-between conductive leads and undergoes a change in resistance when the material sorbs a volatile analyte. In these embodiments, the change in electrical resistance between the leads can be correlated to the sorption of a volatile analyte to the sensor material. Additional examples of metal organic framework (MOF) materials and their use in sensors can be found in U.S. Pat. Nos. 8,735,161, 8,480,955, and International Application No. PCT/US2015/049402, which are hereby incorporated by reference in their entirety.

In some embodiments, the sensing materials for photonic, field effect transistor (FET), and nanogenerator-based sensors can include conducting and non-conducting polymeric networks. In some embodiments, the polymeric networks can be cross-linked (e.g., hydrogels and elastomers) and non-cross-linked. In some embodiments, these materials can undergo changes in optical properties (e.g., due to a refractive index change), electrical properties (e.g., conductance), phase transitions and physical dimensions (e.g., upon swelling/contraction and a consequent change in refractive index or resistance) in response to sorption of one or more volatile analytes, and which can be analyzed to characterize the analyte. Non-limiting examples of polymers that can form the polymeric network according to some embodiments include polyaniline, polypyrrole, polythiophene, poly(phenylene sulphide-phenyleneamine), perylene tetracarboxylic diimide, polyurethane, polystyrene, poly(methyl methacrylate), polyacrylate, polyalkylacrylate, substituted polyalkylacrylate, polystyrene, poly(divinylbenzene), polyvinylpyrrolidone, poly(vinylalcohol), polyacrylamide, poly(ethylene oxide), polyvinylchloride, polyvinylidene fluoride, polytetrafluoroethylene, and other halogenated polymers, hydrogels, organogels, and combinations thereof. In some embodiments, the polymers can include random and block copolymers, branched, star and dendritic polymers, and supramolecular polymers. In some embodiments, the polymers can include one or more natural materials, such as cellulose, natural rubber (e.g., latex), wool, cotton, silk, linen, hemp, flax, feather fiber, and combinations thereof.

In some embodiments, the sensitivity (i.e., detection limit) of the sensor can be less than 5 ppm, with detectable refractive index change of up to ˜10⁻⁷.

In some embodiments, the sensor is a photonic crystal. In some embodiments, the photonic crystal can be a porous photonic crystal (PPC). In some embodiments, the porous photonic crystal can be a 1-dimensional porous photonic crystal, 2-dimensional porous photonic crystal, or 3-dimensional porous photonic crystal.

In some embodiments, the sensor is a field effect transistor. For the electronic sensing according to these embodiments, the gate material for the field-effect transistor (FET) or the material of the nanogenerator electrodes can include one or more micro- and mesoporous layers that permit adsorption of the analyte of interest. In some embodiments, the porous layer can be chemically functionalized, and this functionalization, together with the pore geometry, can collectively affect the diffusion rates of gas or vapor into or within the pores. In some embodiments, the pore geometry, layer thickness, porosity, and surface functionalization can be varied, individually or collectively, to obtain a desired sensitivity to an analyte of interest. In some embodiments, the pore geometry, layer thickness, porosity, and surface functionalization can be varied, individually or collectively, to affect biological kinetics of a sample. Non-limiting examples of field-effect transistors and methods of tuning their sensitivity to an analyte of interest can be found, for example, in International Patent Application No. PCT/IB2007/051764, which is hereby incorporated by reference in its entirety.

In some embodiments, the sensor is a nanogenerator. Non-limiting examples of nanogenerators include surface-acoustic-wave-actuated piezo-electric nanogenerators or triboelectric photonic nanogenerators. Additional non-limiting examples of nanogenerator-based sensors can be found in U.S. Pat. No. 9,595,894, the contents of which are hereby incorporated by reference in their entirety.

In some embodiments, the field-effect transistor (FET) or nanogenerator sensing material can comprise non-porous materials, such as conducting polymers, which exhibit physical changes, e.g., a change of conductance, when exposed to different chemicals. In some embodiments, the gate electrode layer can comprise metals such as Ta, Fe, W, Ti, Co, Au, Ag, Cu, Al, or Ni, or organic materials such as PSS/PEDOT or polyaniline. In some embodiments, the gate electrode material is chosen such that it is a good conductor. In some embodiments, the first dielectric layer can comprise amorphous metal oxides such as Al₂O₃ and Ta₂O₅, transition metal oxides such as HfO₂, ZrO₂, TiO₂, BaTiO₃, SrTiO₃, BaZrO₃, PbTiO₃, and LiTaO₃, rare earth oxides such as Pr₂O₃, Gd₂O₃, and Y₂O₃, or silicon compounds such as Si₃N₄, SiO₂ and microporous layers of SiO and SiOC. In some embodiments, the first dielectric layer can comprise polymers such as SU-8, BCB, PTFE, or even air. In some embodiments, the source electrode and the drain electrode can be fabricated using metals such as aluminium, gold, silver or copper or, alternatively, conducting organic or inorganic materials. In some embodiments, the organic semiconductor can comprise materials selected from poly(acetylene)s, poly(pyrrole)s, poly(aniline)s, poly(arylamine)s, poly(fluorene)s, poly(naphthalene)s, poly(p-phenylene sulfide)s or poly(phenylene vinylene)s. In these embodiments, the semiconductor also may be n-doped or p-doped to enhance conductivity. In some embodiments, the second dielectric layer can include the same materials listed for the first dielectric layer. In some embodiments, the second dielectric layer also shields the layers below from outside conditions, therefore waterproof coatings such as PTFE or silicones may be used in these embodiments.

In some embodiments, the sensor can include an organic semiconductor. Non-limiting examples of organic semiconductors according to one or more embodiments include pentacene, anthracene, rubrene, phthalocyanine, CC, CO-hexathiophene, α-dihexylquaterthiophene, α-dihexylquinquethiophene, α-dihexylhexathiophene, bis(dithienothiophene), dihexyl-anthradithiophene, n-decapentafluorophenylmethylnaphthalene-1-tetracarboxylic diimide, Ceo CeO infused organic polymers, poly(9,9-dioctylfluorene-alt-benzothiadiazole) (F8BT), poly(p-phenylene vinylene), poly(acetylene), poly(thiophene), poly(3-alkylthiophene), poly(3-hexylthiophene), poly(triarylamines), oligoarylamines, poly(thienylenevinylene), and combinations thereof.

In some embodiments, mesoporous sensing materials for photonic, field effect transistor (FET), and nanogenerator-based sensors can be fabricated by alternating spin-, dip-, or spray-coating of nanoparticle suspensions of materials with a high refractive index contrast. Non-limiting examples of materials with high refractive index contrast, in accordance with some embodiments, include silica, alumina, iron oxide, zinc oxide, tin oxide, alumina silicates, aluminum titanate, beryllia, noble metal oxide, platinum group metal oxide, titania, zirconia, hafnia, molybdenum oxide, tungsten oxide, rhenium oxide, tantalum oxide, niobium oxide, vanadium oxide, chromium oxide, scandium oxide, yttria, lanthanum oxide, ceria, thorium oxide, uranium oxide, and other rare earth oxides, and combinations thereof. In some embodiments, such colloidal nanoparticle suspensions can be synthesized by wet-chemistry methods, e.g., sol-gel hydrolysis.

In some embodiments, the separation distance between the inlet 104 and the sensor 108 can be varied to tune the sensitivity of the device for analyzing gasses and gas mixtures.

In some embodiments, the photonic crystals can be a thin film on a transparent substrate, e.g., glass, the shape of which can be, for example, flat, round, spherical, and the like.

In some embodiments, multiple sensors or sensor arrays, each with its own response to the sniffing sequences can be placed within the chamber, and their combined effect can be analyzed.

### Detection Time of the Sensor

In some embodiments, the detection time of the sensor 108 depends upon the configuration of the device 100, including, for example, the position of the sensor 108 on or in the chamber 102, the speed of the injection into the inlet 104, the volume of the gas injected, the possibility of gas leakage from the chamber 102, and the porosity and surface chemistry of the sensor 108.

Furthermore, in some embodiments, the kinetics discussed above can be tuned by the temperature of the device. In some embodiments, the temperature can increase slowly or in steps.

### Sensor Response

In some embodiments, the plurality of sensor responses can include a spectral response. In some embodiments, the spectral response can include a bandgap shift. In some embodiments, the plurality of sensor responses (e.g., spectral responses or bandgap shift) can be detected using a spectrometer or a spectrophotometer.

In some embodiments, the plurality of sensor responses can include a color change. In some embodiments, the color change can be detected using a camera. In some embodiments, the camera can be a smartphone camera. In these embodiments, the color change detected by the camera can be converted into a spectral response. In these embodiments, these images can be converted to an RGB color model, which can in turn be converted to a HSV color model. In these embodiments, the wavelength corresponding to each color present in the HSV color model can be estimated, which can provide the spectral shift.

In some embodiments, the plurality of spectral responses can include contour plots, wavelength derivative plots, Fourier amplitude and phases, and their derivatives, histogram of gradients, wavelet transforms, and a combination thereof.

### Sniffing Sequences

The present disclosure utilizes active, dynamic sampling of a gas or mixture of gasses to improve analysis results. Sniffing sequences can be used to actively sample the gas being analyzed. In accordance with some embodiments, sniffing sequences are provided through a pattern of actions, wherein the pattern of actions may be either pre-defined, optimized or determined through machine learning. Sniffing sequences can include those pre-defined based on experiments, machine learning methods, or human intuition. Sequences may be generated using e.g., artificial intelligence or machine learning methods such as supervised, semi-supervised, self-supervised, and unsupervised methods (including methods based on federated learning) and used for e.g., classification, regression, clustering, etc. Examples of sniffing features or actions include the following:

(1) inhale, wherein inhale comprises introducing the gas into the chamber, typically injecting the gas through the inlet, wherein the inlet and outlet are both open;

(2) wait, wherein wait comprises allowing the sample gas concentration to decrease slowly, typically by opening the outlet without injecting any gas;

(3) exhale, wherein exhale comprises cleansing the sensor, such as by flushing the chamber to remove the sample gas, typically by injecting a carrier gas or mixture of gases into the chamber through the inlet, wherein the inlet and outlet are both open;

(4) hold, wherein hold comprises maintaining a relatively constant atmosphere in the chamber, typically by holding the gas in the chamber with both the inlet and outlet closed;

(5) convect, wherein convect comprises circulating the gas in the chamber, typically with both the inlet and outlet closed; and

(6) de-pressurize or vacuum, wherein the pressure in the chamber is reduced.

A sniffing recipe can be used to control the concentration of odor in the sensor chamber. As the sensor (e.g., photonic Bragg stack) is exposed to the vapors of a particular compound, the adsorption of the vapors into a thin film causes the reflectance of the Bragg stack to shift towards redder colors which is caused by the increase in the effective refractive index in the porous layers of the Bragg stack. This redshift can be recorded using a spectrophotometer. The evolution of the shift of the reflectance peak over time can be quantified using the phase of a Fourier transform. The shift of the spectrum can be shown using the phase (instead of the phase derivative) to show the increase in the adsorption in the Bragg crystal. Because the vapors are injected directly into the chamber, the color of the crystal changes rapidly and experiments rely predominantly on the adsorption and desorption kinetics of different vapors. Focusing on adsorption and directing the injection of the vapors straight at the photonic crystal accelerates the analysis substantially.

In some embodiments, some basic sniffing patterns include, but are not limited to, the following: (1) Fast and short sniffing 110, where the odor is inhaled in a number of short bursts before exhaling, and (2) deep sniffing 120, where the odor is inhaled in a single breath. During fast sniffing, short (e.g., 1 second) intervals of inhaling are interrupted by 0.5 second intervals of waiting, allowing the odor to distribute in the chamber. After a total of 5 inhales and waits, the odor is exhaled for 5 seconds as nitrogen flushes the chamber and sensor (e.g., Bragg stack). By interrupting the flow of odorant into the chamber, the concentration at the sensor periodically decreases and never fully equilibrates. Instead, the phase approaches and then oscillates around a dynamic mean between adsorption maximum and desorption minimum.

In deep sniffing, the odor may be inhaled for a long period of time before any of the other actions such as wait, pressurize, exhale. In accordance with a non-limiting example the inhaling period can be a 5 second inhale followed by a 2.5 second wait—before the odor is again exhaled for 5 seconds. These values are representative only and can be varied as warranted by the particular analysis being conducted. For example, the inhale period can be any multiple or single sequences of lengths such as 1 second, 5 seconds, 10 seconds, 50 seconds, 100 seconds, 5 minutes, or 10 minutes. As a result, the total amount of odorant adsorbed onto the sensor during deep sniffing is likely higher.

In short sniffing, the odor may be inhaled for a relatively shorter period of time before any sequence of actions such as wait, pressure, exhale or even inhale are performed. A non-limiting example is a short sniff for 0.5 seconds can be followed by a 1 second wait. This sequence can be repeated for a specified number of times (e.g., 5) in succession, before finally a long exhale (e.g., 5 seconds). This inhale period can be any multiple or single sequence of lengths such as 0.1 seconds, 0.5 seconds, 1 seconds, 2 seconds, 5 seconds, or 15 seconds.

The sequences of sniffing steps in FIG. 1A produce a time-dependent signal that can be analyzed as shown in FIG. 1B using any combination of signal processing, machine learning, regression, segmentation, feature extraction to determine the desired form of result (classification, regression, prediction, etc.). Furthermore, the results from one sequence can be used to inform subsequent sequences to further analyze the odors. Subsequent sequences can be chosen from a list of pre-defined sequences or generated using e.g., artificial intelligence algorithms including e.g., recurrent neural networks, convolutional neural networks, transformers, generative adversarial networks, auto encoders, and natural language processing algorithms more broadly. This results in an adaptive sensor that mimics the sniffing behavior in animals to track and identify odors. Such a sequence of sniffing features or actions can result in better estimates of the composition or properties of a gas or mixture, increased confidence in the predicted results, or monitor the development of a situation over time (e.g., tracking a building fire by changes in vapor composition).

As shown in FIG. 1C, the sequence of sniffing sequences, and the sniffing sequences themselves, can be pre-defined, optimized, or free sniffing. Pre-defined sequences may be pre-determined by the manufacturer or user as a set of recipes that specify the injection sequences or the sequence of sniffing sequences or both, and generally used for the detection of a specific compound (e.g., for medical diagnostics). Optimized sniffing sequences allow some flexibility in the length, repetition, order, or other elements of certain or all segments of a sniffing recipe and allowing some flexibility to optimize the sniffing sequence to a specific sample, based on the analysis of the previous sniffing sequence. In free sniffing, the sequence and length of segments are fully determined by artificial intelligence or other machine decision mechanisms to build a new sequence for a specific sample. This allows the most flexibility in exploring the chemical and physical properties of a sample.

Specific parameters relating to the sniff pattern will depend on the configuration of the sampling device, the dynamics of the sensor itself, and the properties of the pump or other device (e.g., vacuum) used to move the sample through the chamber. Accordingly, these parameters can vary significantly in consideration of these other variables. In accordance with some embodiments, the following values may be considered as general guides:

short inhale or wait step: 0.01-3 seconds; 0.1-1 seconds; 0.5-1 seconds

deep inhale or long wait step: 1-30 seconds; 3-15 seconds; 5-10 seconds

exhale: 0.5-300 seconds; 1-20 seconds; 3-10 seconds.

Of course, as noted above these values should not be considered limiting as they will depend on the particular device and samples being tested.

In accordance with some embodiments, the disclosed sniffing sequences can be associated with other methods/variation other than temporal changes. Long and short sniffs aim to modulate the integration time of the signal. However, other sniff patterns such as low pressure sniffs vs. high pressure sniffs can give key information on the physical properties of the sample (evaporation rate, diffusivity, etc.) and its signature dependence on pressure; classification accuracy can be increased using this method.

In accordance with some embodiments, the disclosed sniffing sequences can include but are not limited to low-concentration vs high-concentration sniffs. In accordance with this aspect, the sniffing of the sample may be filtered. In accordance with certain embodiments, the filter may be a physical mesh disposed between the sample and sensor. The filter can either allow the gas to permeate through the mesh to provide for complete sensing or the filter can be used to resist the gas from reaching the sensor in which a low-concentration sniff is maintained. This can be useful for probing over-saturated signals without changing time integration of the sniff sequence. In accordance with other aspects, the filter can be selectively permeable in which the sensor can get information on particular analytes at a time. A non-limiting example includes where the sample produces gases A, B, C. In accordance with this example, a first mesh allows gas A to permeate easily, ˜50% of B to permeate (or a slow permeation), and completely resists the permeation of gas C. A second mesh enables gas C to permeate completely, but resists permeation of A, and B. This unique permeation signature through the filter between the sample and sensor allows the signal to be partially de-convoluted. This has capabilities to strengthen machine learning classification.

In accordance with one embodiment, the filter can be a size exclusive mesh that prevents gas analytes above certain size from traversing. In accordance with another embodiment, the filter that may operate based on van der Waals or polarity to prevent certain partially charged gases from transporting through the filter. Filters can be used to filter non-polar analytes from polar analytes during sensing or to filter smaller molecules. In accordance with one particular example, filters can be used to facilitate distinguishing nitrogen gas, ammonia, and putrescine. All three compounds described are nitrogen containing compounds and can be detected by a sensor sensitive to nitrogen containing compounds. However, only putrescine among these is a compound signatory of spoilage of food, whereas other nitrogen containing compounds are merely byproducts of other processes (for example, N₂ is a carrier gas). Thus, being able to use selective filter to suppress the dominant compound (N₂, a carrier gas) can be useful to discern more useful smells of putrescine that can be low in concentration but the compound of interest.

FIG. 1D lists some non-limiting examples for potential applications of an adaptive sensor as disclosed herein that utilizes a pattern of sequences (sniffing) to determine simple and complex odors, such as indoor, industrial, medical, environmental sensing, safety and personal applications.

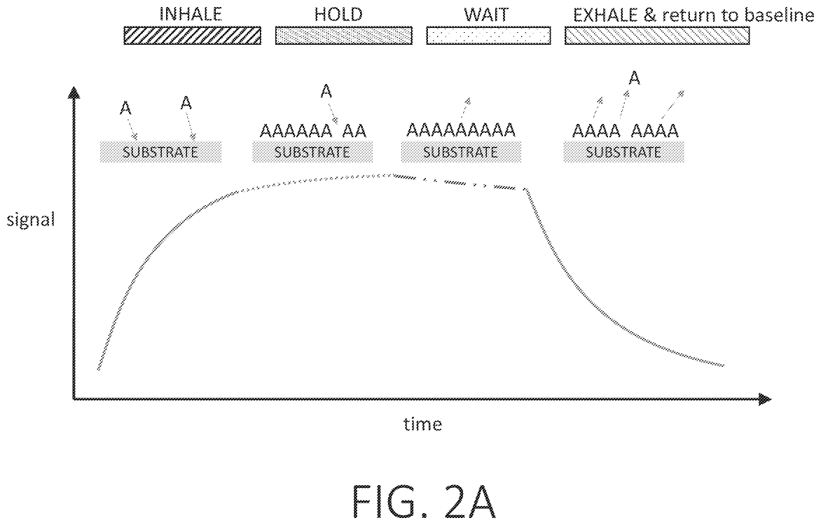

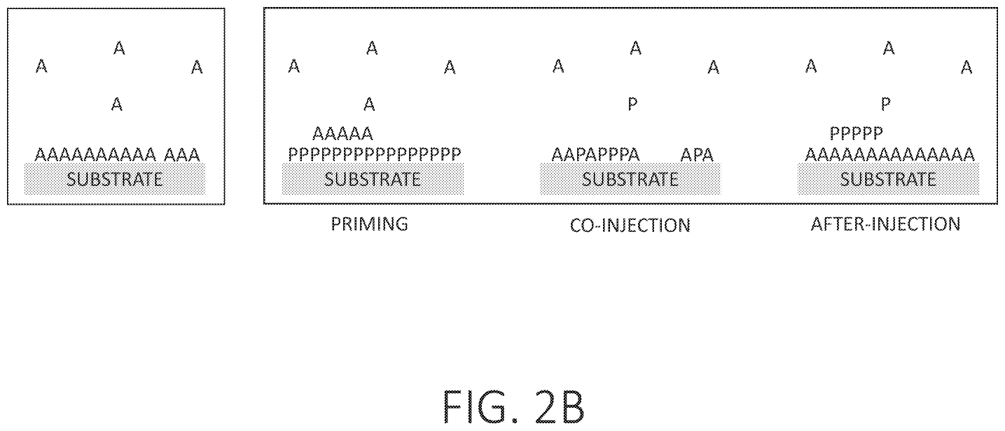

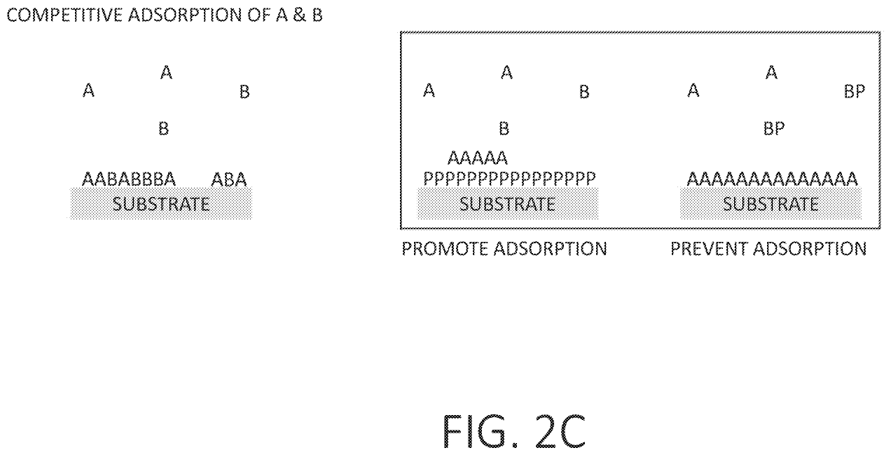

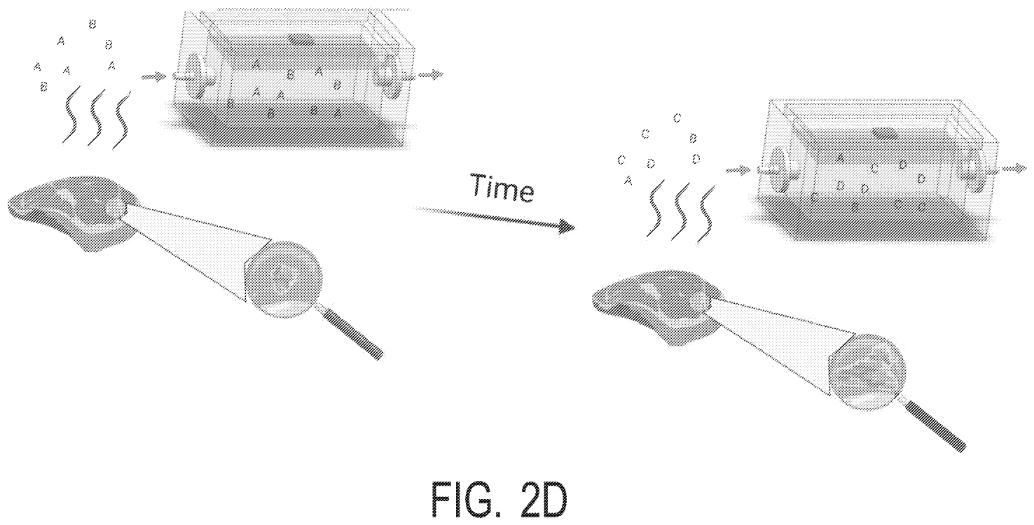

FIG. 2A illustrates a possible sensor signal to a set of simple sniffing steps and the corresponding adsorption behavior characteristic of some embodiments. As the concentration of gasses or vapors in the sensor chamber increases during inhale, the increase in the sample leads to an increase in the sensor signal. In sensor devices involving adsorption of gasses or vapors onto the surface, this process continues during hold, when the vapor concentration inside the chamber is held constant, and decreases during wait, when the gasses or vapors are slowly escaping through the outlet. During exhale, the sample gasses or vapors are replaced by injecting a reference gas or by applying a vacuum and the sensor response returns back to the baseline response enabling repeated use of the sensor for the same or other samples.

As exemplified in FIG. 2B, the sensor response to a tested component A is further modified through the use of primer gases P. Using a second gas P to inject before (Priming), during (Co-injection), or after the sample is injected (After-injection), allows the modification of the surface to promote or limit the adhesion or interaction of certain types or portions of a sample, thus affecting the adsorption/desorption dynamics. Primer gases can include simple chemicals or monomers/polymers that, for example, change the hydrophilicity or hydrophobicity of the sensor or walls of the chamber, biochemical reagents like antibodies or RNA-strands that match specific markers in the sample, e.g., using click-chemistry, or physical modification by introducing e.g., ions, plasmas, or heat.

As exemplified in FIG. 2C, priming with a second gas or co-injection of a second gas can include chaperone molecules P that can bind to a target gas species A, thus facilitating its adsorption and affecting its adsorption/desorption kinetics, or bind to certain unwanted components of the gas mixture B, and creating species BP that are prevented from adsorption. Such steps provide the way to resolve the issue of a competitive adsorption of A and B that cannot be resolved by simple inhale/exhale sequences of mixed gas samples.

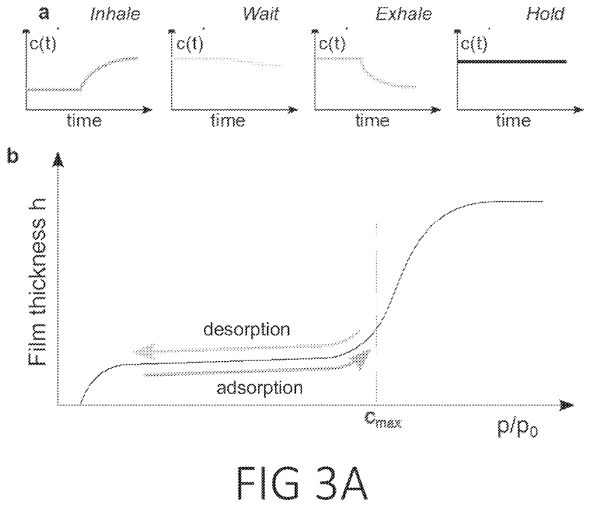

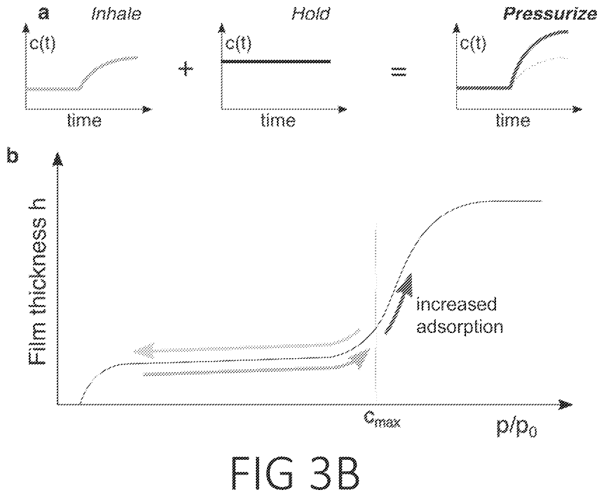

As shown in FIGS. 3A and 3B, the duration and frequency of the sniffing steps can be used to actively change the concentration of gasses or vapors in the sensor chamber and characterize the evolution of the adsorption/desorption behaviors of gasses or vapors by moving up and down the adsorption isotherm. Adsorption isotherms are sensitive to the composition and materials of the sensor which makes the adsorption of gasses or vapors in response to a sniffing sequence that dynamically changes the concentration powerful to distinguish complex mixtures based on the unique features of the evolution of their adsorption profile.

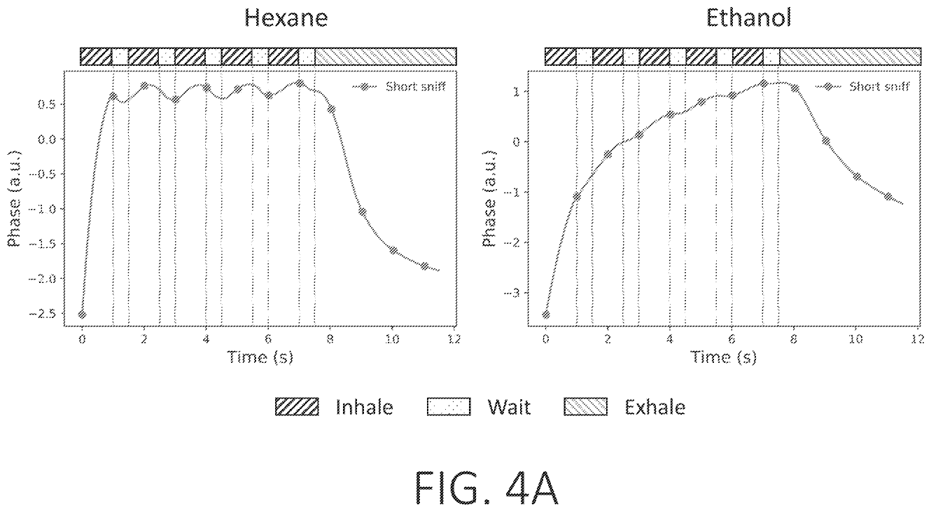

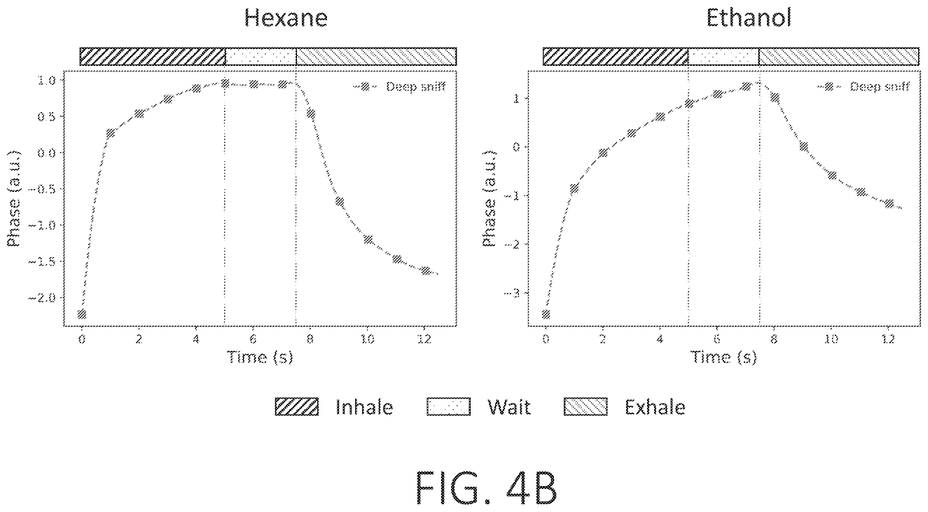

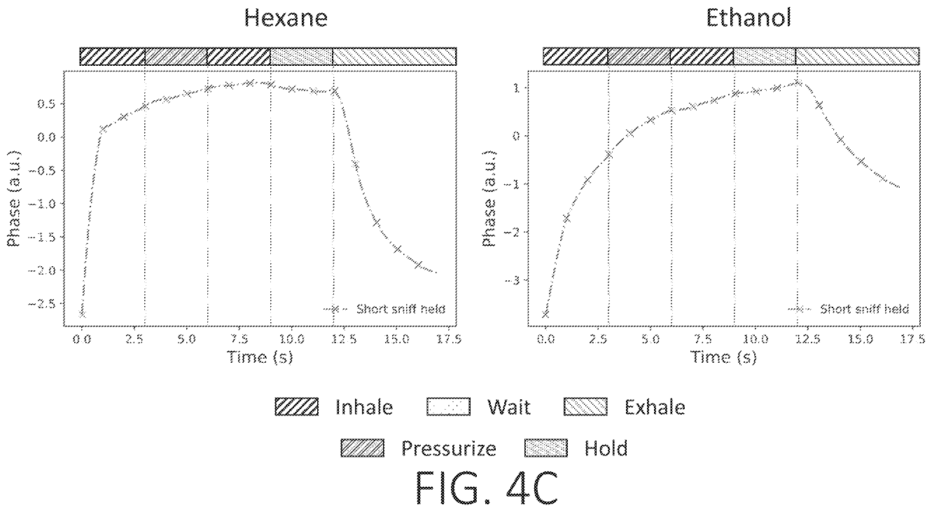

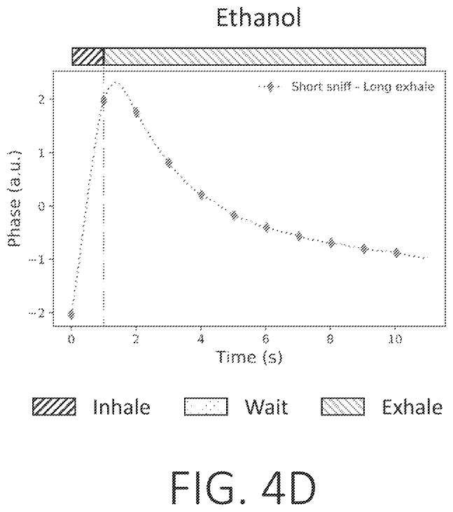

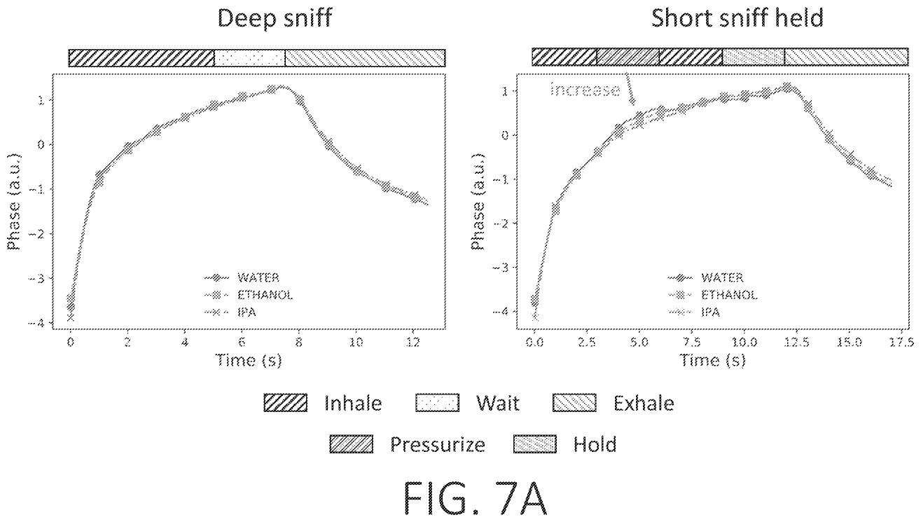

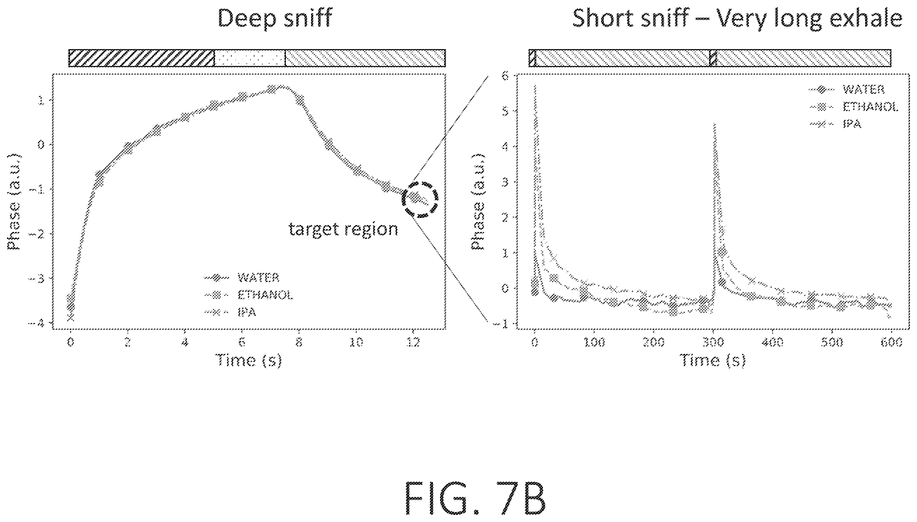

FIG. 4 provides examples of the sensing procedure by illustrating several sniffing sequences that can be used to characterize hexane, an apolar volatile organic compound, and ethanol, a polar volatile organic compound, that are both common in industrial applications. FIG. 4A shows fast sniffing where five bursts of inhale at one second each are interrupted by 0.5 second duration wait leading to an oscillatory signal that is useful to distinguish quickly adsorbing chemicals from less quickly adsorbing chemicals as seen by the amplitude in hexane versus ethanol. In FIG. 4B, deep sniffing which instead of short bursts of inhale, uses one inhale at five seconds, followed by 2.5 seconds of wait which is useful for slowly adsorbing chemicals. FIGS. 4C and 4D show two more recipes that can be used to focus on certain sections of fast sniffing and deep sniffing. In FIG. 4C, pressurize is used to increase the effect of adsorption to differentiate highly similar chemicals (see FIG. 7A), and FIG. 4D shows a long exhale that focuses on the desorption process in fast or deep sniffing (see FIG. 7B).

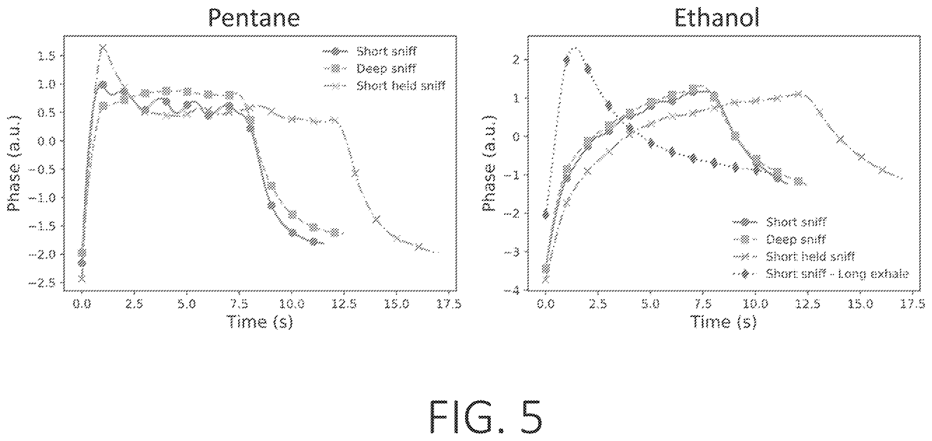

FIG. 5 provides a comparison of the sniffing recipes in FIG. 4 for pentane, a non-polar volatile organic compound similar to hexane, and ethanol showing that different sniffing sequences can produces categorically and characteristic sensor responses. The sniffing sequences are shown in FIGS. 4A-4D. More specifically, the sequences used are: Short sniff (5 repetitions of: 1 s inhale, 0.5 s wait, followed by 5 s exhale); Deep sniff (5 s inhale, 2.5 s wait, 5 s exhale); Short sniff held (3 s inhale, 3 s pressurize, 3 s inhale, 5 s exhale); Short sniff-Long exhale (1 s inhale, 10 s exhale).

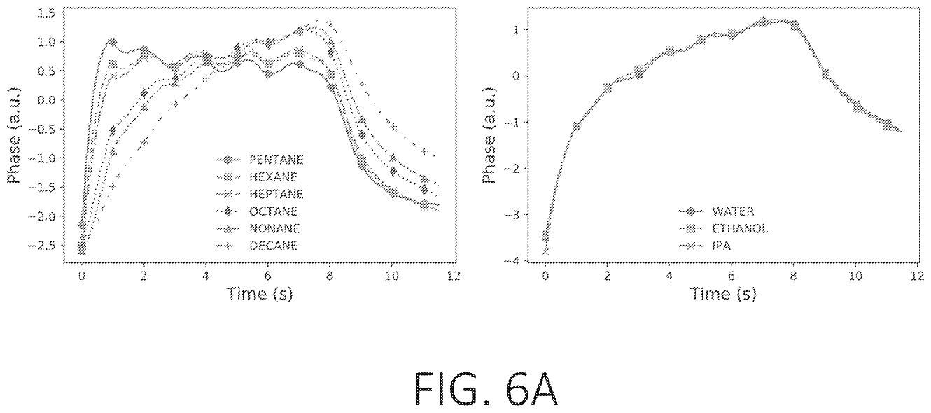

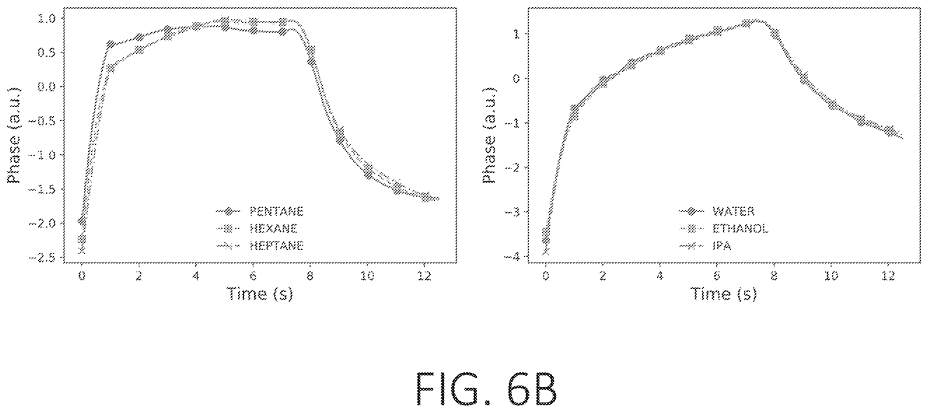

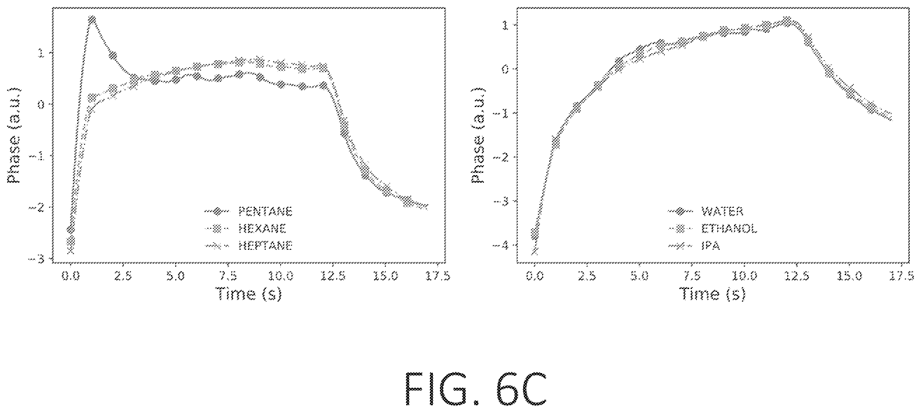

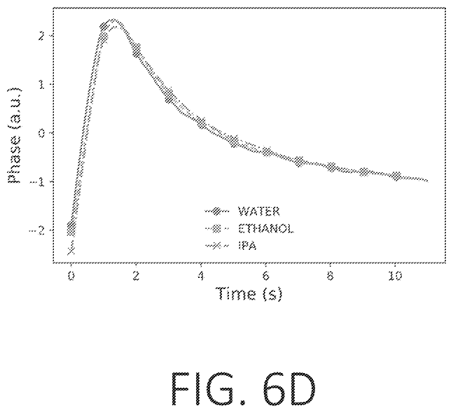

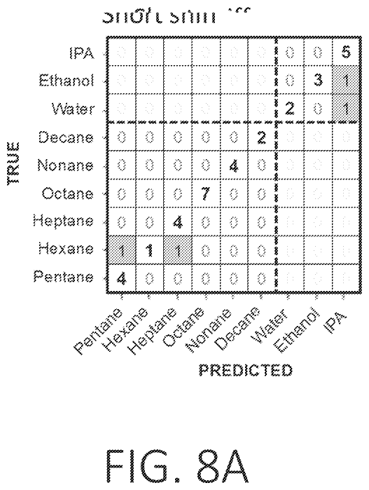

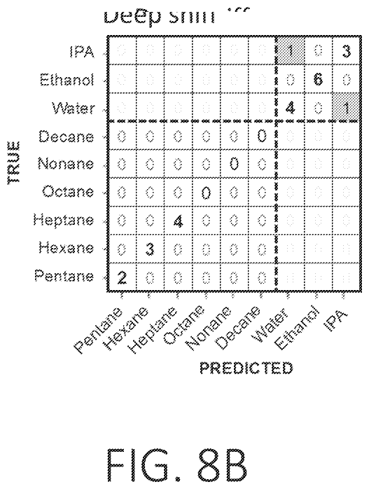

FIGS. 6A to 6D are phase curves showing the sensor response to various apolar (left) and polar (right) chemicals using different sniffing recipes: short sniff (5 repetitions of: 1 s inhale, 0.5 s wait, followed by 5 s exhale) (FIG. 6A), deep sniff (5 s inhale, 2.5 s wait, 5 s exhale) (FIG. 6B), short sniff held (3 s inhale, 3 s pressurize, 3 s inhale, 5 s exhale) (FIG. 6C), and short sniff-long exhale (1 s inhale, 10 s exhale) (FIG. 6D). In addition to providing characteristic sensor responses for different chemicals, by using different sniffing sequences one can more easily distinguish certain types of chemicals than others, which could be used to isolate target vapors from background signals using sniffing. Confusion matrices for the results in FIG. 6A and FIG. 6B are provided in FIG. 8A and FIG. 8B, respectively. The confusion matrices show that different sniffing recipes are better at distinguishing certain kinds of vapors than others.

Different sniffing recipes can be used to increase the ability of the sensor to differentiate between various chemicals, e.g., by using pressurize to increase the rate of adsorption and thereby increase the contrast between different chemicals (FIG. 7A). Also, in FIG. 7B, increasing or decreasing the duration of the sniffing segments of a sniffing recipe can be used to target small differences in portions of the sensor response to provide a stronger contrast.

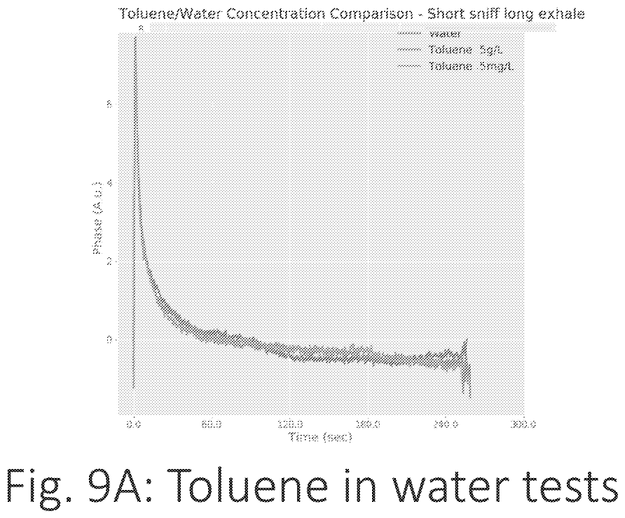

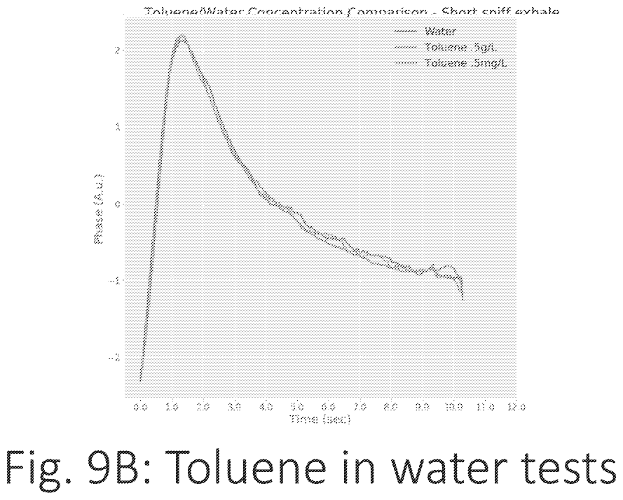

Phase signal for low-concentration toluene in water mixtures using two sniffing sequences are shown in FIG. 9A (short sniff-long exhale) and FIG. 9B (short sniff-exhale). These results demonstrate the potential of the method and device disclosed herein to be used for the detection of asymptomatic Malaria carriers with a characteristic change in body odor involving toluene.

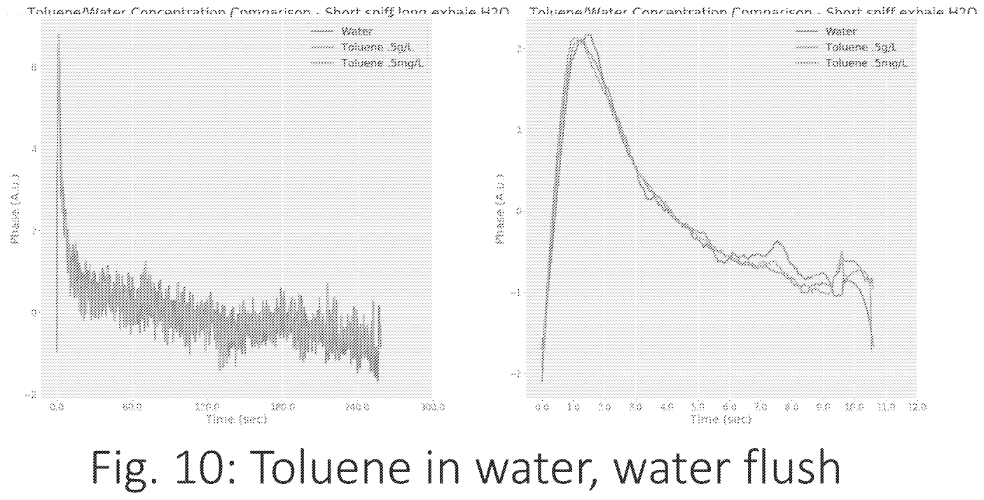

Phase signal for low-concentration toluene in water mixtures using two sniffing sequences with a different baseline gas containing water vapor to isolate the signal for toluene are provided in FIG. 10 (left plot—short sniff-long exhale) (right plot—short sniff-exhale).

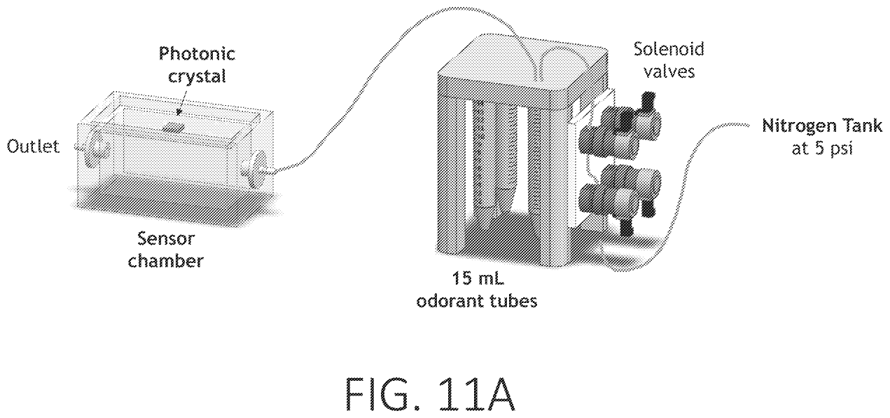

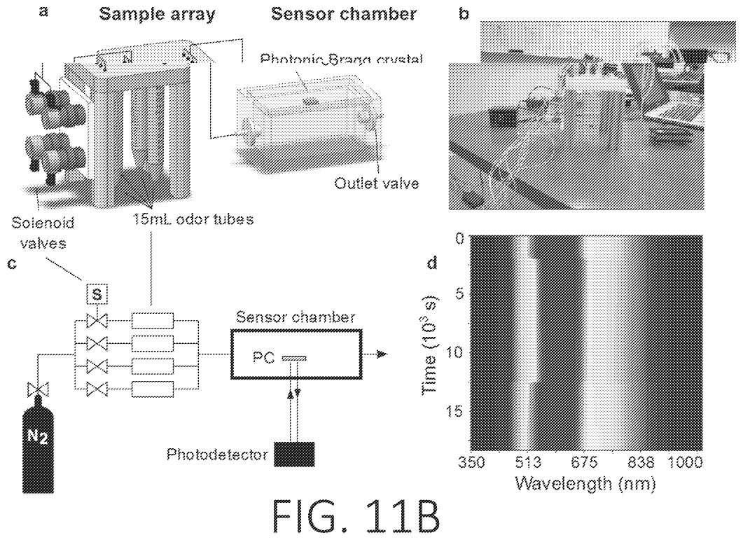

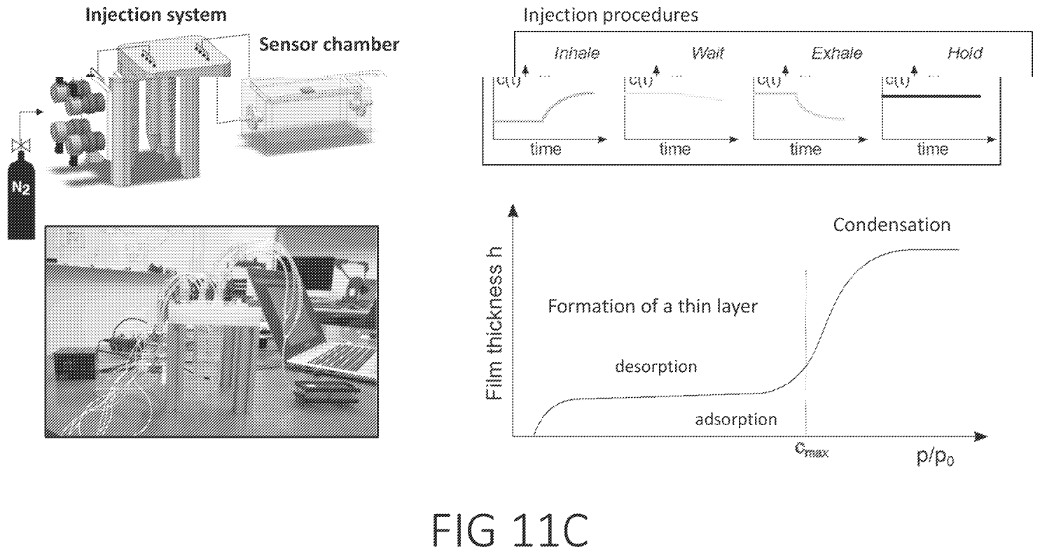

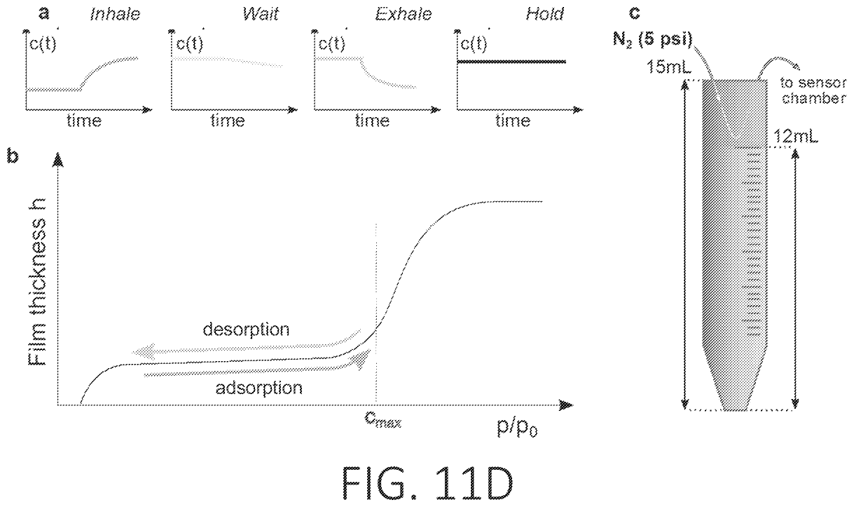

FIG. 11A shows a sensor device wherein solenoid valves and pressurized gas are used to inject gases according to sniffing recipes or patterns of actions, according to one or more embodiments.

FIG. 11B (a) shows a sensor device wherein solenoid valves, pressurized gas and liquid filled tubes are used to inject gases according to sniffing recipes or patterns of actions, according to one or more embodiments. Solenoid valves control odor injection into the sensor chamber containing the photonic crystal. (b) is a photograph of the built sensor setup. (c) is a schematic of the sensor design. (d) provides the reflectance spectra recorded over long exposure time to illustrate maximum strength of the sensor signal.

FIG. 11C shows another arrangement for a sensor device wherein solenoid valves, pressurized gas and liquid filled tubes are used to inject gases according to sniffing recipes or patterns of actions, according to one or more embodiments.

FIG. 11D illustrates that sniffing procedures control kinetics of vapor adsorption. (a) Illustrations of the effective odor concentration using different sniffing procedures. Sniffing can be used to increase (inhale), decrease (wait, exhale), or hold (hold) the concentration of the odor in the chamber. (b) Adsorption isotherm for a general odor molecule. The amount of adsorbed analyte is a function of the concentration of odor in the chamber and is limited by the odor concentration in the sampled air. As shown in (c), since vapors are injected from the atmosphere above a 12 mL liquid aliquot in a 15 mL Falcon tube, the concentration of the injected odorant depends on its volatility. Less volatile compounds will therefore be injected at a lower concentration as would be the case in a real-world scenario.

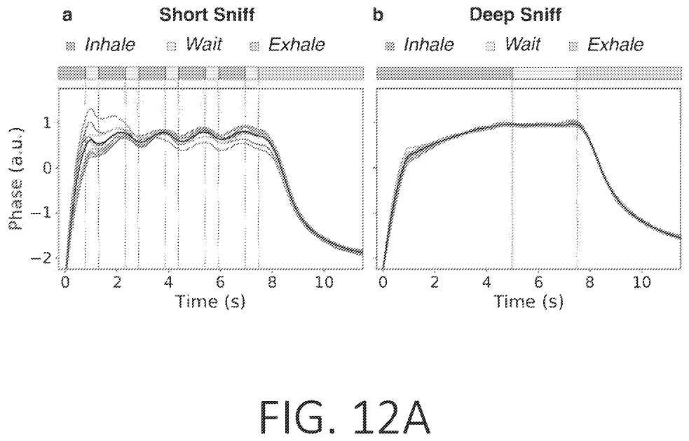

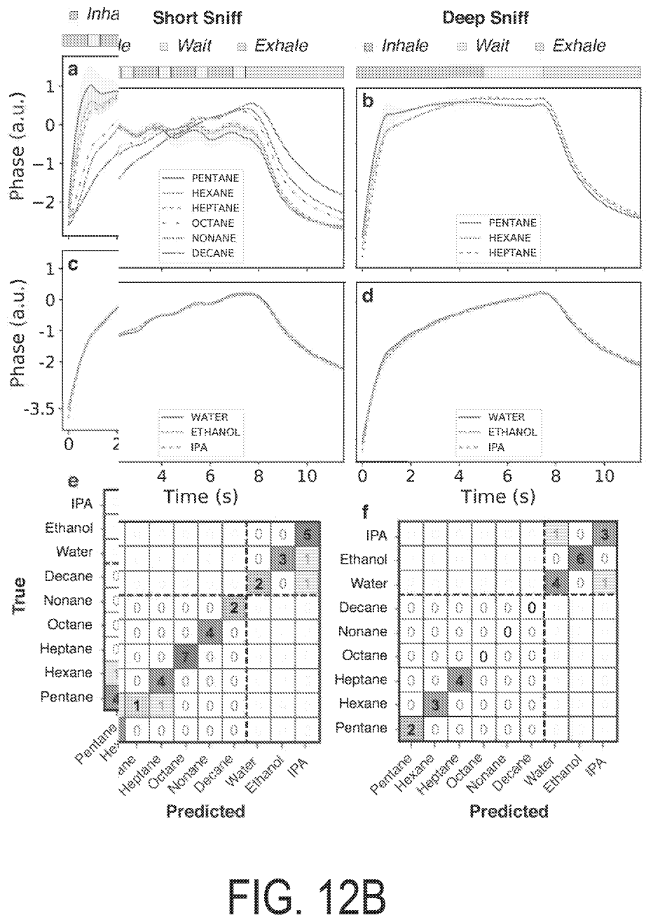

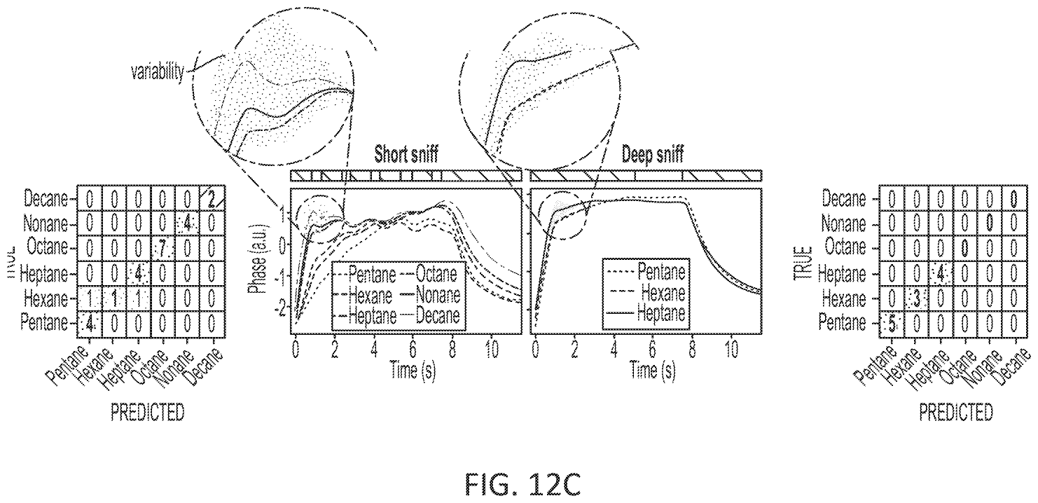

FIG. 12A shows a signal sensitivity comparison for short sniff and deep sniff showing the diverse reaction to fast and slow sniffing sequences, according to one or more embodiments. (a) Short sniff: Hexane vapor is sampled 5 times using 1 s inhales with 0.5 s waits in-between, and then exhaled for 5 s. The spectral shift quickly saturates and continues to oscillate. (b) Deep sniff: Hexane is inhaled for 5 s, followed by a 2.5 s wait and 5 s exhale. The phase continues to slowly increase during the inhale but shows less variability.

FIG. 12B shows an analysis of short sniff and deep sniff sequences on apolar and polar chemicals using support vector machine classification (machine learning), according to one or more embodiments. Sniffing procedures are shown to control kinetics of vapor adsorption/desorption. Sniffing procedures used to detect different sample odors including alkanes and some polar compounds using short (a,c) and deep sniffing (b,d). Confusion matrices for sniffing of sample odorants using short (e) and deep sniff (f) showing good numbers of correct predictions (indicated by colored (diagonal) elements) for both sniffing procedures.

FIG. 12C shows a detailed comparison of increased sensitivity of different sniffing sequences for apolar chemicals using short and deep sniff, according to one or more embodiments.

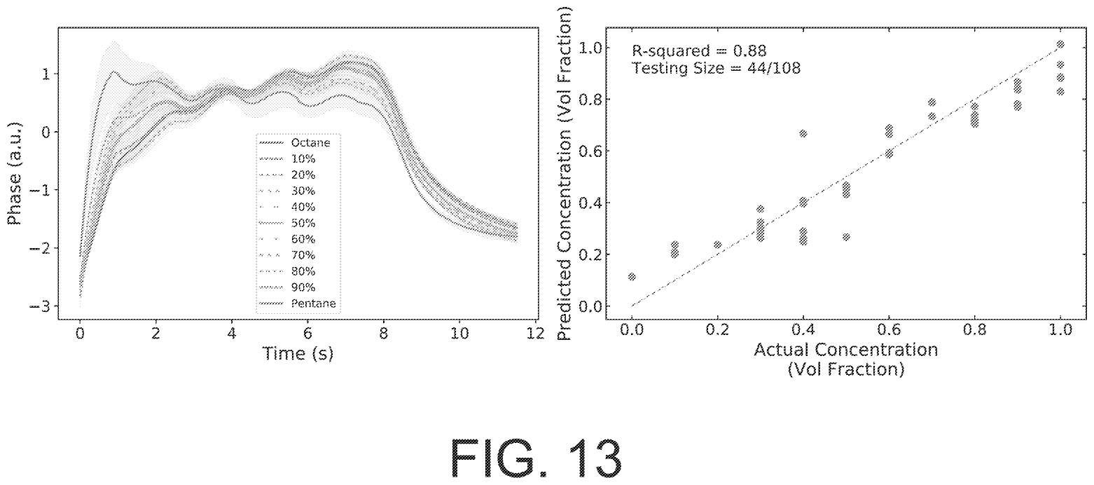

FIG. 13 shows a binary mixture concentration analysis using support vector regression, according to one or more embodiments.

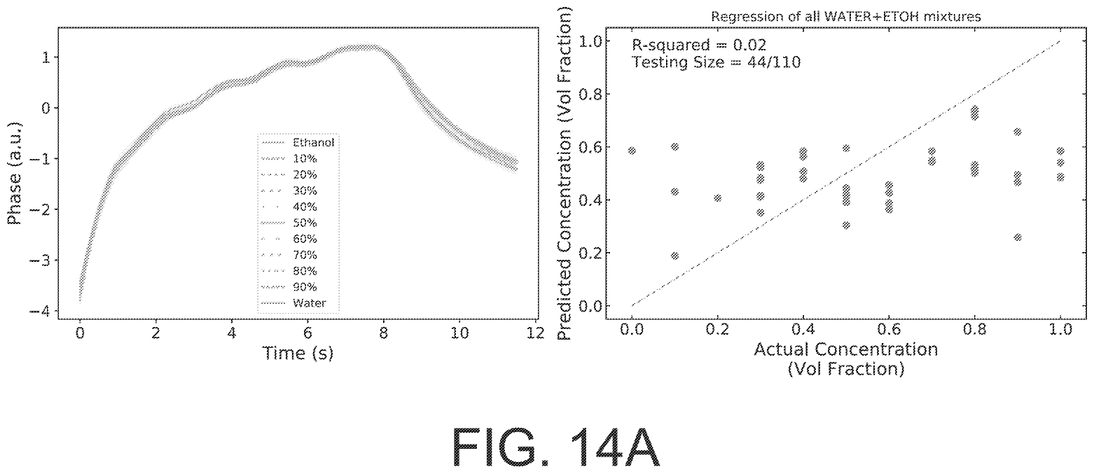

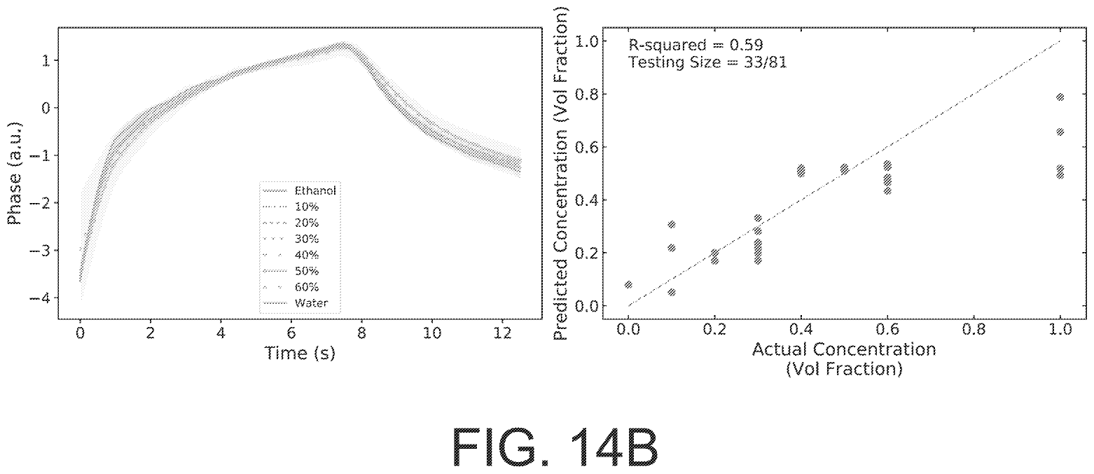

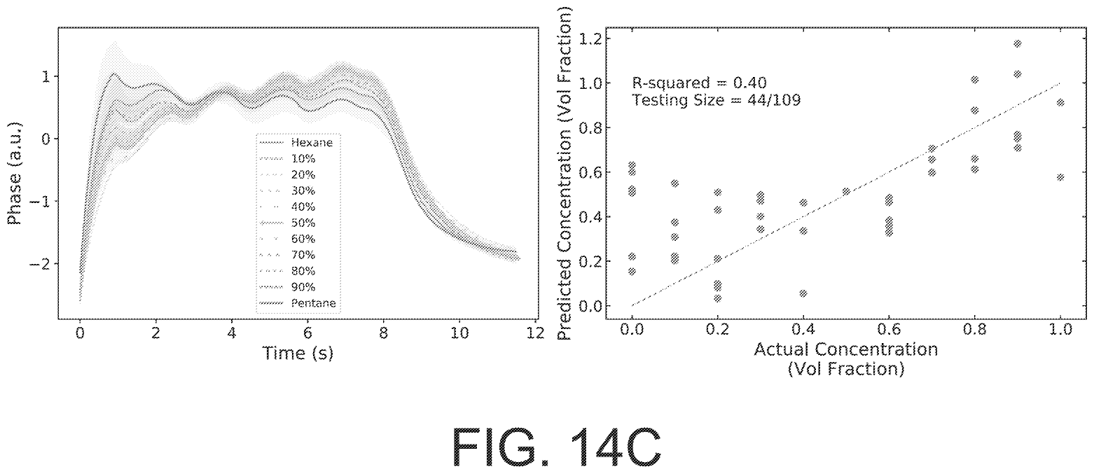

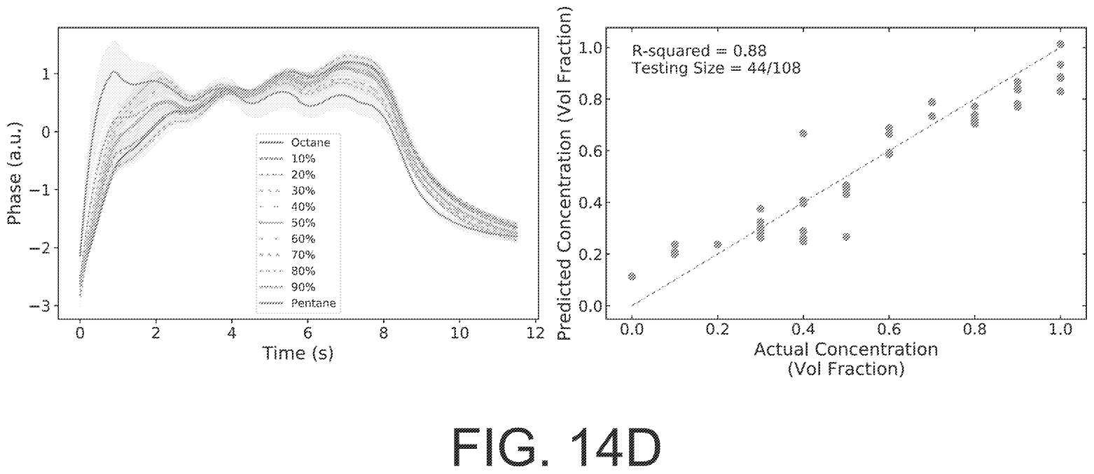

FIGS. 14A (short sniff sequence) and 14B (deep sniff sequence) show concentration analysis of binary mixtures of ethanol and water using different sniffing techniques showing that the use of sniffing can significantly improve the accuracy of the sensor, according to one or more embodiments.

FIG. 14C shows an analysis of the mixing ratio of binary mixtures of pentane-hexane, according to one or more embodiments.

FIG. 14D shows an analysis of the mixing ratio of binary mixtures of pentane-octane, according to one or more embodiments.

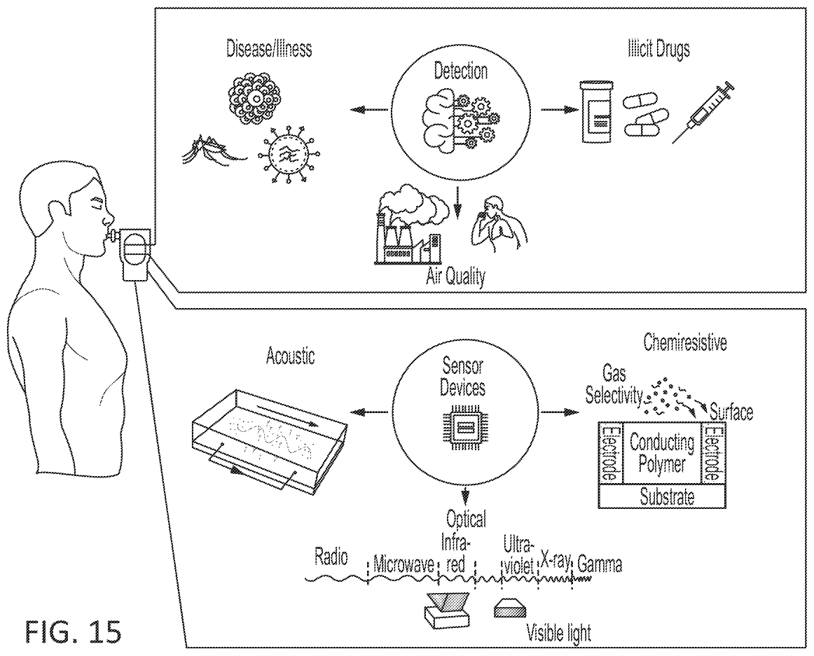

FIG. 15 illustrates various sensing elements and sensing outputs in accordance with certain aspects of the present disclosure. The application of a device such as a breathalyzer can extended to sensing things other than blood alcohol levels. With this sniffing sequences and technology, various types of sensing elements can be used such as acoustic sensors, surface acoustic wave (SAW) devices, optical sensors, such as photonic crystals, and chemiresistive thin films, such as organic thin film transistors (OTFTs). The device although depicted as a breathalyzer can be any hand-held device, including smart phones (with sniffing capabilities), smart watches, etc.

In certain embodiments, the sniffing element may be any of optical, acoustic, temperature, pressure, chemical, pH, type sensors or any combination thereof.

In certain embodiments, the sniffing element may be part of a larger device in which the device is a smart mobile device (either smart phones, or smart watches) in which the smart device is equipped with at least one of the sniffing elements to detect any of diseases, illnesses, air quality, illicit drugs consumption, or any other blood adsorbed compounds that can emit volatile compounds in breath.

In certain embodiments, the device may be used by persons to monitor progression of diseases, monitor self-health, or perform non-invasive evaluation of certain compounds.

In certain embodiments, the device can be used by law enforcement personnel to detect use of illicit drugs to prevent illegal activities. In other embodiments, the same device can be used by health professionals to diagnose consumption of illicit drugs of unconscious patients to improve health treatment.

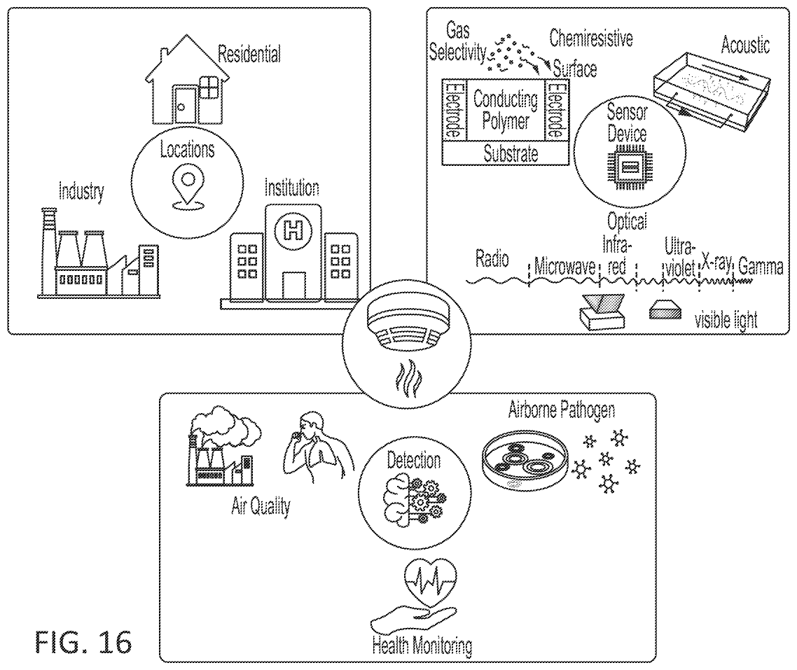

FIG. 16 illustrates an indoor sensor which may be used to provide sniffing technology to indoor spaces in locations such as residential homes, industrial areas, or institutional buildings. The sensing elements can be of various forms and nature (e.g., acoustic sensors, surface acoustic wave (SAW) devices, optical sensors, such as photonic crystals, and chemiresistive thin films, such as organic thin film transistors (OTFTs)). The indoor sensor can be used to detect indoor air quality, airborne pathogens, and space monitoring for health concerns.

In certain embodiments, the sensing device can be of the form of a smart device in which the data is seamlessly transmitted to a continuously monitored server.

In certain embodiments, the sensing is used to monitor closed spaces for unsafe environments such as development of asphyxiation hazards, or dangerous chemicals.

In certain embodiments, the sensor can be deployed to detect spread of biological compounds.

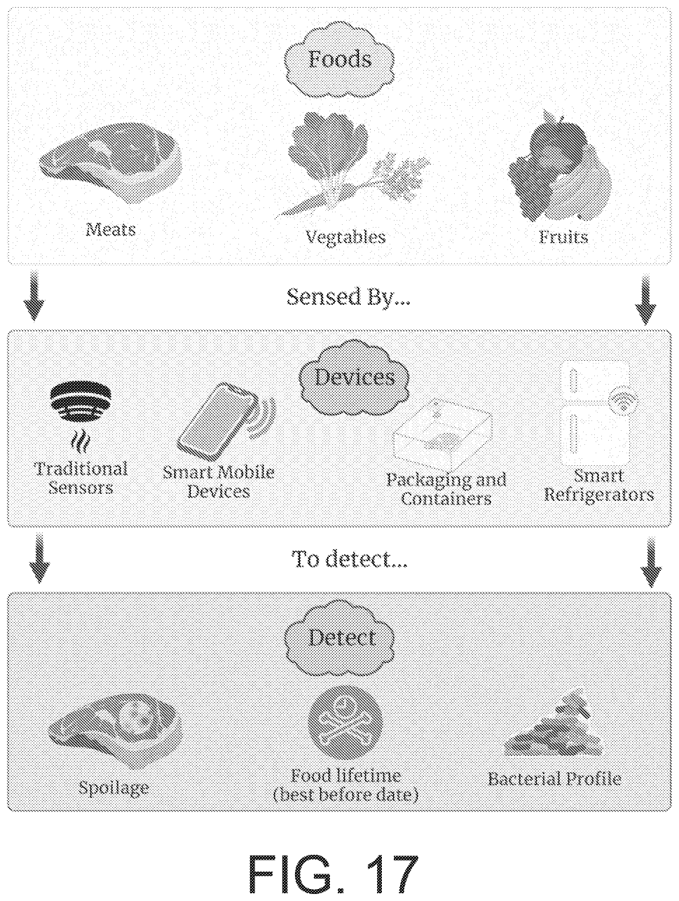

FIG. 17 illustrates various applications of sniffing technology to biological samples (foods). These samples can be sensed by either traditional sensors, smart mobile devices, packaging, or smart refrigerators to detect spoilage, food lifetime, and bacterial profile.

The sniffing sequences can be used on biological samples, which themselves are evolving in time. The biological samples can be food items such as meats, vegetables, fruits, or even dairies products. These samples can be detected via various device forms such as traditional indoor sensors, smart mobile sensors, packaging or container sensors, or smart refrigerator sensors. These sensors can detect for spoilage of contents, predict expected lifetime (best before date) of foods, or even profile the current bacterial content within the food.

In certain embodiments, the sniffing device can be used to diagnose development of harmful bacteria such as Salmonella on meats.

In certain embodiments, the sniffing device can be used to detect spoilage of any food item using odor signatures from bacteria development and their by-products.

In certain embodiments, the sniffing device can be a hand-held mobile smartphone in which the sensing can provide a quick method to probe a food item before consumption.

In certain embodiments, the sniffing device can be part of the food packaging in which the state of the food can be evaluated from the packaging (such as RFID scan), or a color indicator on the wrapping.

In certain embodiments, the sniffing device can be part of a container in which various food devices can be sensed and detected by a universal database on the cloud or server. In these embodiments, more usage of such a device strengthens the sensing capabilities and accuracy of the device as each use by consumer is tracked and added as training points for machine learning purposes.

In certain embodiments, the sniffing device can be setup in a particular 3-dimensional arrangement within a smart refrigerator, or a storage unit, that allows of sensing multiple foods together. In such an embodiment, the 3-dimensional configuration of various sensors allows the detection of spoilage of a particular food within the fridge.

In certain embodiments, the smart applications of such sniffing devices can be used to predict estimated lifetime of food items; the time before the consumption of such a food item poses no/or minimal health concerns.

In certain embodiments, the sniffing device can be used at the industrial scale to ensure healthy food is maintained in stock. In other industrial applications, the device may serve to profile bacterial species and concentrations. In such embodiments, the use of this device can be used for food engineering purposes such as probiotic foods. In a society where nutritional and dietary choices are becoming more accessible to consumers, the use of such device can be used to probe bacterial content and engineer foods with heathy bacteria to improve the overall health of consumers and specifically gut microbiome.

### Analysis of Gas Mixtures Using Machine Learning

In some embodiments, the disclosed methods and devices for analyzing gasses or gas mixtures via detecting the time evolution of the plurality of dynamic responses uses data acquisition and analysis routines. In these embodiments, the data acquisition and analysis routines can lead to a high dimensionality, i.e., the number of possible independent variables, of the sensing platform, which was not possible with other single-output and combinatorial steady-state sensors, and which can be implemented to perform the compositional analysis of analytes that are not included in a data library (i.e., “unknowns”) via supervised and unsupervised machine learning frameworks (MHLFs).

In some embodiments, the machine learning frameworks facilitate the characterization and classification of single-component and gas mixtures, as well as the recognition of specific components, for example through the formation of a library of sensor responses. In some embodiments, the use of an array of photonic structures or field effect transistors (FETs) with the same or different porosities and surface functions can enhance the accuracy and precision of the machine learning methods. In some embodiments, various machine learning (or “self-learning”) algorithms can be implemented to perform classification, regression, and clustering tasks. In some embodiments, various machine learning (or “self-learning”) algorithms can, in part, enable analysis of the composition of the gas mixture. Non-limiting examples of the machine learning algorithms include supervised machine learning algorithms, unsupervised machine learning algorithms, semi-supervised machine learning algorithms, support vector machines, transfer learning neural networks, and segmented regression algorithms. Additional machine learning algorithms include, but are not limited to, artificial intelligence algorithms including e.g., recurrent neural networks, convolutional neural networks, transformers, generative adversarial networks, auto encoders, and natural language processing algorithms more broadly. Still other machine learning algorithms that may be used include, but are not limited to, reinforcement learning (e.g., policy iteration, value iteration, SARSA method, Q-Learning, Deep Q learning and any off-policy or on-policy variations with or without neural networks), support vector regression, probabilistic models, mixture models, topic models, inference bayes networks, hidden Markov models, clustering models, K-Means and Hierarchical Agglomerative Clustering (HAC).

In some embodiments, the experimentally obtained data can first be pre-processed to extract nuanced independent features from the plurality of spectral responses (e.g., via contour plots, Fourier transform amplitudes and phases and their derivatives, wavelength derivative plots, histogram of gradients, or wavelet transforms), and then imported into a classifier, e.g., a support vector machine or principal components analyzer, or a regressor (e.g., linear, radial basis function, LASSO, or ridge support vector regressors) with optimized performance, to perform pattern recognition and discrimination of the composition of the volatile liquid mixture. In some embodiments, monitoring the response of a photonic sensor can be performed using a spectrometer or a camera and converting the recorded data into color models. In some embodiments, monitoring the response of a field effect transistor (FET) sensor or nanogenerator can be performed through measuring the current-voltage signal or the time-dependent current change. In some embodiments, the obtained profiles can be further processed and combined into data vector for further classification.

In some embodiments, the choice of machine learning framework can vary as a function of the application. In some embodiments, where signal processing is performed, to obtain a list of features from the measured data, support vector machines are a useful first choice. In these embodiments, support vector machines can be used for classification of analytes into hazard classification, compound classes, or based on other features using support vector classifiers. In addition, in these embodiments, support vector regressors are suitable for analyses of concentration ranges and physical parameters. In some embodiments, more specialized classification and regression algorithms, such as bagging classifiers, can be useful to, for example, divide the dataset for further analysis or segment a range of mixtures into smaller regression ranges. In some embodiments the sensor data is used without post-processing. In these embodiments, advanced machine learning frameworks, such as neural networks, transfer learning, and deep neural networks, are useful. In these embodiments, transfer learning, in particular, can be applied to improve sensor accuracy with limited datasets. Examples of transfer learning that can be used include, but are not limited to, zero-shot, one-shot, and few-shot learning.

### Examples

### Machine Learning Frameworks for Classification and Regression

In some embodiments, machine learning frameworks constitute a useful analysis tool for the non-equilibrium sensing method according to one or more embodiments described herein. In some embodiments, the machine learning framework includes a series of sensor signal-preprocessing methods (e.g., transform and normalization), followed by extraction and selection of the sensor features from the initial multidimensional fingerprints, and followed by classification, regression, clustering, and cross-validation. In some embodiments, if the analyte is not from the training data set and the supervised classification/regression is not possible, the machine learning framework can establish an unsupervised model for mapping the unknown fingerprint with the target physico-chemical properties. Examples of the machine learning frameworks according to some embodiments include LASSO, kernel ridge regression, support vector machine, neural networks (including transfer learning neural networks), GANs, decision trees, bagging classifiers, multiclass logistic regression, principal component analysis, and linear discriminant analysis.

It will be appreciated that while one or more particular materials or steps have been shown and described for purposes of explanation, the materials or steps may be varied in certain respects, or materials or steps may be combined, while still obtaining the desired outcome. Additionally, modifications to the disclosed embodiment and the invention as claimed are possible and within the scope of this disclosed invention. For example, different sensor types and configurations, surface treatments, measurement parameters, data analysis techniques, and other aspects discussed above can be combined in various combinations to tune the apparatus to particular analytes or characteristics to measure, as will be apparent to those of skill in the art.

1. A method for analyzing a gas, the method comprising:
providing a chamber comprising an inlet, an outlet and a sensor;
introducing the gas into the chamber;
controlling a concentration of the gas in the chamber according to a sniffing recipe, wherein the sniffing recipe comprises a sequence of actions and the sniffing recipe is either pre-defined, optimized or determined through machine learning, wherein the sniffing recipe comprises:
(1) inhale, wherein inhale comprises introducing the gas into the chamber; and at least one of the following actions:
(2) exhale, wherein exhale comprises cleansing the sensor;
(3) wait, wherein wait comprises allowing a concentration of the gas to decrease slowly; and
detecting, over time and by the sensor, a characteristic indicative of a compound or compounds present in the gas.

2. The method of claim 1, wherein the sniffing recipe comprises a pattern of actions.

3. The method of claim 2, wherein the pattern of actions comprises a specified length of time for each action in the sequence.

4. The method of claim 1, wherein the sniffing recipe further comprises one or more of the following actions: (4) hold, wherein hold comprises maintaining a relatively constant atmosphere in the chamber; (5) pressurize, wherein pressurize comprises increasing the pressure in the chamber; (6) convect, wherein convect comprises circulating the contents of the chamber; (7) de-pressurize or vacuum, wherein de-pressurize or vacuum comprises reducing the pressure in the chamber; (8) priming, wherein priming comprises introducing a known compound to the chamber before introducing the gas; (9) co-injection, wherein co-injection comprises introducing a known chaperone compound simultaneously with the gas; and (10) after-injection, wherein after-injection comprises introducing a compound that affects the desorption of the gas after introducing the gas to the chamber.

5. The method of claim 1, wherein the sniffing recipe comprises a plurality of inhale actions alternating with a plurality of hold actions.

6. The method of claim 1, wherein the sniffing recipe comprises a pattern of actions and a specified length of time for each action in the pattern, wherein the sequence of actions and specified length of time are pre-defined.

7. The method of claim 6, wherein the pre-defined pattern of actions is based on the gas being analyzed.

8. The method of claim 1, wherein the sniffing recipe comprises a first recipe followed by a second recipe, wherein said first recipe is pre-defined and said second recipe is determined based on machine learning from measurements resulting from the first recipe.

9. The method of claim 1, further comprising priming the chamber with a known compound prior to introducing the gas being analyzed.

10. The method of claim 1, further comprising introducing a known compound simultaneously with the gas being analyzed.

11. The method of claim 1, further comprising introducing a known compound after introducing the gas being analyzed.

12. The method of claim 1, wherein the sensor comprises a photonic crystal.

13. The method of claim 1, wherein the sensor comprises a field-effect transistor (FET).

14. The method of claim 1, wherein exhale comprises flushing the chamber with another fluid to remove the gas being analyzed.

15. The method of claim 14, wherein flushing the chamber with another fluid comprises injecting the fluid through the inlet.

16. A device comprising:
a chamber configured to receive a gas to be analyzed, the chamber comprising an inlet and an outlet;
a sensor disposed in the chamber, the sensor configured to detect a characteristic indicative of a compound or compounds present in the gas; and
a pump configured to operate in accordance with a sniffing recipe, wherein the sniffing recipe comprises a sequence of actions and the sniffing recipe is either pre-defined, optimized, determined through machine learning, or a combination thereof, wherein the sniffing recipe comprises:
(1) inhale, wherein inhale comprises activating the pump to introduce the gas into the chamber; and at least one of the following actions:
(2) exhale, wherein exhale comprises flushing the chamber to remove the gas
(3) wait, wherein wait comprises allowing a concentration of the gas to decrease slowly.

17. The device of claim 16, wherein the sniffing recipe further comprises one or more of the following actions: (3) wait, wherein wait comprises allowing a concentration of the gas in the chamber to decrease slowly; (4) hold, wherein hold comprises holding the gas in the chamber; (5) pressurize, wherein pressurize comprises increasing the pressure in the chamber to sample a larger section of the adsorption isotherm; (6) convect, wherein convect comprises circulating the contents of the chamber; (7) de-pressurize or vacuum, wherein de-pressurize or vacuum comprises reducing the pressure in the chamber; (8) priming, wherein priming comprises introducing a known compound to the chamber before introducing the gas; (9) co-injection, wherein co-injection comprises introducing a known chaperone compound simultaneously with the gas; and (10) after-injection, wherein after-injection comprises introducing a compound that affects the desorption of the gas after introducing the gas to the chamber.

18. The device of claim 16, wherein the sensor is selected from the group consisting of a photonic crystal, a field effect transistor, a nanogenerator, and photomechatronic nanostructures.

19. The device of claim 18, wherein the sensor comprises a photonic crystal.

20. The device of claim 16, wherein the sensor provides a spectral response.

21. The device of claim 20, wherein the spectral response comprises a bandgap shift.

22. The device of claim 20, further comprising a spectrophotometer configured to detect the evolution of the spectral response in time.

23. The device of claim 16, further comprising at least one processor configured to run one or more machine learning algorithms on data provided by the sensor, the machine learning algorithm capable of determining a pattern of actions based on features of the data from the sensor, wherein at least one of the one or more machine learning algorithms comprises at least one of pattern recognition, classification, regression, and segmented regression.

24. The device of claim 23, wherein the one or more machine learning algorithms are selected from the group consisting of LASSO, kernel ridge regression, decision trees, bagging classifiers, multiclass logistic regression, principle component analysis, linear discriminant analysis, supervised machine learning, semi-supervised machine learning, non-supervised machine learning, support vector machines, transfer learning neural networks, segmented regression, or a combination thereof.

25. The device of claim 16, wherein the pump or a second pump is configured to introduce a known compound(s) into the chamber in accordance with one or more of the following:
1) prior to introducing the gas being analyzed;
2) simultaneously with the gas being analyzed;
3) after introducing the gas being analyzed.

26. The device of claim 16, wherein the pump or a second pump is configured to introduce an unknown compound(s) into the chamber in accordance with one or more of the following:
1) prior to introducing the gas being analyzed
2) simultaneously with gas being analyzed
3) after introducing the gas being analyzed.

27. The device of claim 16, further comprising a filter disposed in the chamber between the inlet and the sensor.

28. The device of claim 27, wherein the filter comprises a size exclusive mesh.

29. The device of claim 16, wherein the device is selected from the group consisting of an indoor sensor, a medical diagnostic device, a food quality sensor, an air quality sensor and combinations thereof.

30. The device of claim 16, wherein the gas being analyzed is from a biological sample.

31. A device comprising:
chamber configured to receive a gas to be analyzed, the chamber comprising an inlet; and
a sensor disposed in the chamber, the sensor configured to detect a characteristic indicative of a compound or compounds present in the gas;
wherein said device is configured to operate in accordance with a sniffing recipe, wherein the sniffing recipe comprises a sequence of actions and the sniffing recipe is either pre-defined, optimized, determined through machine learning, or a combination thereof, wherein the sniffing recipe comprises:
(1) inhale, wherein inhale comprises introducing the gas into the chamber; and at least one of the following actions:
(2) exhale, wherein exhale comprises flushing the chamber to remove the gas;
(3) wait, wherein wait comprises allowing a concentration of the gas to decrease slowly.

32. The device of claim 31, wherein said device is a handheld device.

33. The device of claim 31, wherein the device is a breathalyzer, smart phone or smart watch.

34. The device of claim 31, wherein the gas to be analyzed is a user's breath.

35. The device of claim 34, wherein the device instructs the user to breathe in accordance with the sniffing recipe.

36. The device of claim 31, wherein the sniffing recipe further comprises one or more of the following actions: (3) wait, wherein wait comprises allowing a concentration of the gas in the chamber to decrease slowly; (4) hold, wherein hold comprises holding the gas in the chamber; (5) pressurize, wherein pressurize comprises increasing the pressure in the chamber to sample a larger section of the adsorption isotherm; (6) convect, wherein convect comprises circulating the contents of the chamber; (7) de-pressurize or vacuum, wherein de-pressurize or vacuum comprises reducing the pressure in the chamber; (8) priming, wherein priming comprises introducing a known compound to the chamber before introducing the gas; (9) co-injection, wherein co-injection comprises introducing a known chaperone compound simultaneously with the gas; and (10) after-injection, wherein after-injection comprises introducing a compound that affects the desorption of the gas after introducing the gas to the chamber.
